(19) United States
(12) Patent Application Publication
(10) Pub. No.: US 2026/0327706 A1
(43) Pub. Date: Oct. 22, 2026

(54) COMPOSITIONS AND METHODS FOR MONITORING AND SELECTING THERAPIES

(71) Applicant: The Regents of the University of Michigan, Ann Arbor, MI (US)

(72) Inventors: Lonnie Shea, Ann Arbor, MI (US); Ravi Raghani, Ann Arbor, MI (US); Kelly Arnold, Ann Arbor, MI (US); Katarina DiLillo, Ann Arbor, MI (US)

(21) Appl. No.: 19/113,220

(22) PCT Filed: Sep. 28, 2023
(86) PCT No.: PCT/US2023/033959

§ 371 Date: Mar. 19, 2025

Related U.S. Application Data
(60) Provisional application No. 63/412,125, filed on Sep. 30, 2022.

(51) Int. Cl. C12Q 1/6886; G01N 33/57515

(52) U.S. Cl. CPC C12Q 1/6886; G01N 33/57515; C12Q 2600/158

(57) ABSTRACT

The present disclosure relates to gene panels, materials, and methods for evaluation of cancer in a subject. In particular, provided herein are synthetic scaffolds and methods of use thereof for predicting or monitoring response to an immune checkpoint inhibitor in a subject.

### STATEMENT REGARDING RELATED APPLICATIONS

This application claims priority to U.S. Provisional Patent Application No. 63/412,125, filed Sep. 30, 2022, the entire contents of which are incorporated herein by reference for all purposes.

### STATEMENT REGARDING FEDERALLY SPONSORED RESEARCH OR DEVELOPMENT

This invention was made with government support under CA243916 awarded by the National Institutes of Health. The government has certain rights in the invention.

### FIELD

The present disclosure relates to compositions, systems, and methods for evaluation of cancer in a subject. In particular, provided herein are synthetic scaffolds and methods of use thereof for predicting or monitoring response to an immune checkpoint inhibitor in a subject.

### BACKGROUND

The treatment of triple negative breast cancer (TNBC) with immune checkpoint blockade (ICB) has shown improvements in patient outcomes. In a phase 3 clinical trial of anti-PD-1 (aPD-1) in mTNBC patients, ICB was found to perform similarly as effective, with reduced grade 3-4 adverse events, as compared to the gold standard chemotherapy (Annals of Oncology. 30 (2019) 397-404). Additionally, in the phase 3 trial of neoadjuvant/adjuvant aPD-1 in early-stage TNBC patients, investigators found improved pathological complete responses and reduced disease progression in the patients receiving ICB (Breast Cancer Res Treat. 167(2018 ) 671-686). However, while ICB-sensitive breast cancer patients have incredible responses, the majority of TNBC patients are resistant to ICB. Moreover, existing markers and gene panels for stratifying patients based upon expected response to ICB are lacking. As such, there is a great need for technologies that stratify patients based on ICB-response.

### SUMMARY

In some aspects, provided herein arc methods comprising obtaining a sample from a microenvironment of a synthetic scaffold implanted in a subject, wherein the subject is a candidate for treatment with an immune checkpoint inhibitor or has received treatment with an immune checkpoint inhibitor, and measuring an expression level or amount of at least one RNA, at least one gene, at least one cell type, and/or at least one protein in the sample. In some embodiments, the subject has received treatment with an immune checkpoint inhibitor, and the sample is obtained from the microenvironment of the synthetic scaffold about 1 to about 28 days after receiving the treatment. In some embodiments, the sample is obtained from the microenvironment of the synthetic scaffold about 7 to about 21 days after receiving the treatment. In some embodiments, the subject has or is suspected of having breast cancer. In some embodiments, the breast cancer is triple negative breast cancer (TNBC).

In some aspects, provided herein are methods comprising obtaining a sample from a microenvironment of a synthetic scaffold implanted in a subject, wherein the subject is diagnosed with or at risk of having cancer and wherein the subject has not received treatment with an immune checkpoint inhibitor, and measuring an expression level of a panel of genes in the sample. In some embodiments, the subject has or is suspected of having breast cancer. In some embodiments, the breast cancer is triple negative breast cancer (TNBC). In some embodiments, the panel of genes comprises two or more genes (e.g. at least 2, at least 3, at least 4, at least 5, at least 6, at least 7, at least 8, at least 9, at least 10 genes) selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises at least Slc2a12, Slc5a7, Aldh1a2, and Id3. In some embodiments, the panel of gene comprises less than 50 genes. For example, in some embodiments the panel comprises 6-50 genes, including at least 6 of Pmepa 1, Gm 50439, Tnfsf 9, Gar 1, Foxf2, Crlf 1, Gm 11847, Id3, Aldh1a2, Slc5a7, Gm49484, Rspo2, Hdac1, X2200002j24Rik, Sh3gf3, and Slc2a12.

In some embodiments, the method further comprises providing an immune checkpoint inhibitor to the subject or an alternative cancer treatment to the subject based upon the expression level of the panel of genes. For example, in some embodiments the method comprises providing an immune checkpoint inhibitor to the subject when the expression level of one or more of Pmepa11, Gm50439, Tnfsf9, and Gar1is decreased relative a control level or a cutoff level. In some embodiments, the method comprises providing an immune checkpoint inhibitor to the subject when the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level. In some embodiments, the method comprises providing an alternative cancer treatment to the subject when the expression level of one or more of Pmepa11, Gm50439, Tnfsf9, and Gar1is increased relative a control level or a cutoff level and/or the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level.

In some aspects, provided herein are methods of predicting response to an immune checkpoint inhibitor in a subject. In some embodiments, methods of predicting response to an immune checkpoint inhibitor in a subject comprise obtaining a sample from a microenvironment of a synthetic scaffold implanted in a subject, wherein the subject is diagnosed with or at risk of having cancer and is a candidate for treatment with an immune checkpoint inhibitor, measuring an expression level or amount of at least one RNA, at least one gene, at least one cell type, and/or at least one protein in the sample, and determining whether the subject is likely to be sensitive or resistant to treatment with the immune checkpoint inhibitor based upon the expression level or amount of the at least one RNA, the at least one gene, the at least one cell type, and/or the at least one protein in the sample. In some embodiments, the subject has or is suspected of having breast cancer. In some embodiments, the breast cancer is triple negative breast cancer (TNBC). In some embodiments, the method comprises measuring an expression level or amount of a panel of RNA, a panel of genes, a plurality of cell types, and/or a panel of proteins in the sample. In some embodiments, the subject is diagnosed with or at risk of having cancer and has not received treatment with an immune checkpoint inhibitor. In some embodiments, the method further comprises comparing the expression level or amount of the at least one RNA, at least one gene, at least one cell type, and/or at least one protein in the sample to a control level or a cutoff level. In some embodiments, the method further comprises providing the immune checkpoint inhibitor to the subject determined as likely to be sensitive to treatment with the immune checkpoint inhibitor, or providing an alternative cancer treatment to the subject identified as being likely to be resistant to treatment with the immune checkpoint inhibitor.

In some embodiments, methods of predicting response to an immune checkpoint inhibitor in a subject comprise measuring the expression level of one or more genes (e.g. at least 2, at least 3, at least 4, at least 5, at least 6, at least 7, at least 8, at least 9, at least 10 genes) selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises at least Slc2a12, Slc5a7, Aldh1a2, and Id3. In some embodiments, methods of predicting response to an immune checkpoint inhibitor in a subject comprise measuring the expression level of a panel of genes. In some embodiments, the method comprises determining that the subject is likely to be sensitive to treatment with the immune checkpoint inhibitor when the expression level of one or more of Pmepa11, Gm50439, Tnfsf9, and Gar1is decreased relative a control level or a cutoff level. In some embodiments, the method comprises determining that the subject is likely to be sensitive to treatment with the immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level. In some embodiments, the method comprises determining that the subject is likely to be resistant to treatment with the immune checkpoint inhibitor when the expression level of one or more of Pmepa11, Gm50439, Tnfsf9, and Gar1is increased relative a control level or a cutoff level. In some embodiments, the method comprises determining that the subject is likely to be resistant to treatment with the immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level. In some embodiments, the method comprises providing the immune checkpoint inhibitor to the subject determined as likely to be sensitive to treatment with the immune checkpoint inhibitor, or providing an alternative cancer treatment to the subject identified as being likely to be resistant to treatment with the immune checkpoint inhibitor.

In some aspects, provided herein are methods comprising obtaining a sample from a microenvironment of a synthetic scaffold implanted in a subject, wherein the subject is diagnosed with or at risk of having cancer and wherein the subject has received treatment with an immune checkpoint inhibitor; and measuring an expression level of a panel of genes in the sample; and/or measuring an amount of one or more cell types selected from B-cells, NK-cells, neutrophils, T-cells, and macrophages in the sample. In some embodiments, the sample is obtained from the microenvironment of the synthetic scaffold about 1 to about 28 days after receiving treatment with the immune checkpoint inhibitor. In some embodiments, the sample is obtained from the microenvironment of the synthetic scaffold about 7 to about 21 days after receiving treatment with the immune checkpoint inhibitor. In some embodiments, the method comprises determining whether the subject is sensitive or resistant to treatment with the immune checkpoint inhibitor based upon the expression level of the panel of genes and/or the amount of the one or more cell types in the sample. In some embodiments, the method further comprises providing an alternative treatment to the subject determined to be resistant to treatment with the immune checkpoint inhibitor.

In some embodiments, the panel of genes comprises two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1, two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3, or two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, the panel of genes comprises at least Ripply3, Fdps, Pagr1a, and Stfa2. In some embodiments, the panel of genes comprises at least Crlf1 Gm18362, Sprtn, and Ptgs2. In some embodiments, the panel of genes comprises six or more genes. In some embodiments, the panel of genes comprises 10 or more genes.

In some embodiments, the panel of gene comprises less than 50 genes. For example, in some embodiments the panel of genes comprises at least 6 but less than 50 genes (i.e. 6-50 genes).

In some embodiments, the method comprises measuring an amount of one or more cell types selected from B-cells, NK-cells, neutrophils, T-cells, and macrophages in the sample. In some embodiments, the method comprises providing an alternative cancer treatment to the subject when the amount of B-cells is increased relative to a control level or a cutoff level. In some embodiments, the method comprises providing an alternative cancer treatment to the subject when the amount of NK-cells is increased relative to a control level or a cutoff level. In some embodiments, the method comprises providing an alternative cancer treatment to the subject when a ratio of the amount of neutrophils: T-cells, neutrophils: B-cells, neutrophils: NK-cells, and/or macrophages: NK-cells is decreased relative to a control.

In some aspects, provided herein are methods of monitoring response to treatment with an immune checkpoint inhibitor in a subject. In some embodiments, methods of monitoring response to treatment with an immune checkpoint inhibitor in a subject comprise obtaining a sample from a microenvironment of a synthetic scaffold implanted in a subject; and measuring an expression level or amount of at least one RNA, at least one gene, at least one cell type, and/or at least one protein in the sample. In some embodiments, methods of monitoring response to treatment with an immune checkpoint inhibitor in a subject comprise obtaining a sample from a microenvironment of a synthetic scaffold implanted in a subject; and measuring an expression level or amount a panel of RNA, a panel of genes, a panel or proteins, and/or one or more cell types in the sample. In some embodiments, the subject is diagnosed with or at risk of having cancer and has received treatment with an immune checkpoint inhibitor. In some embodiments, sample is obtained from the microenvironment of the synthetic scaffold about 1 day to about 28 days after receiving the treatment. In some embodiments, the sample is obtained from the microenvironment of the synthetic scaffold about 7 to about 21 days after receiving the treatment. In some embodiments, the subject has or is at risk of having breast cancer. In some embodiments, the breast cancer is triple negative breast cancer.

In some embodiments, methods of monitoring response to treatment with an immune checkpoint inhibitor comprise comparing the expression level or amount of the at least one RNA, at least one gene, at least one cell type, and/or at least one protein in the sample to a control level or a cutoff level. In some embodiments, the method comprises determining whether the subject is sensitive or resistant to treatment with the immune checkpoint inhibitor based upon the expression level or amount of the at least one RNA, at least one gene, at least one cell type, and/or at least one protein in the sample. In some embodiments, the method comprises providing an alternative cancer treatment to the subject identified as resistant to treatment with the immune checkpoint inhibitor. In some embodiments, the subject has or is at risk of having breast cancer. In some embodiments, the breast cancer is triple negative breast cancer.

In some aspects, provided herein are kits. In some embodiments, provided herein are kits comprising reagents for detection of a panel of genes in a sample. In some embodiments, the panel of genes comprising two or more genes (e.g. (e.g. at least 2, at least 3, at least 4, at least 5, at least 6, at least 7, at least 8, at least 9, at least 10 genes) selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the kit is used in a method of predicting response to an immune checkpoint inhibitor in a subject. In some embodiments, the subject has or is at risk of having breast cancer. In some embodiments, the breast cancer is triple negative breast cancer.

In some embodiments, kit comprises reagents for detection of a panel of genes, wherein the panel of genes comprises two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1. In some embodiments, the panel of genes comprises two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the panel of genes comprises two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, panel of genes comprises less than 50 genes. In some embodiments, the kit is used in a method of monitoring response to treatment with an immune checkpoint inhibitor in a subject. In some embodiments, the subject has or is at risk of having breast cancer. In some embodiments, the breast cancer is triple negative breast cancer. In some embodiments, monitoring response to the treatment with the immune checkpoint inhibitor in the subject comprises contacting a sample obtained from a microenvironment of a synthetic scaffold implanted in the subject with the reagents for detection of the panel of gene. In some embodiments, the sample is obtained about 1 day to about 28 days after receiving the treatment with the immune checkpoint inhibitor. In some embodiments, the sample is obtained about 7 days to about 21 days after receiving the treatment with the immune checkpoint inhibitor.

### DESCRIPTION OF THE DRAWINGS

FIGS. 1A-1F show response to Immune Checkpoint Blockade Treatment. FIG. 1A is a schematic representation of tumor cell inoculation and ICB treatment and response. FIG. 1B shows tumor growth following inoculation. FIG. 1C shows mouse survival after tumor cell inoculation. n=10 per group, *p<0.05, and values=mean+SEM. FIGS. 1D-1F show immunohistochemistry of PDL1 expression in the primary tumor of mice. FIG. 1D shows expression 7 days after inoculation in mice before ICB treatment, FIG. 1E shows expression 21 days after inoculation in ICB-sensitive mice, and FIG. 1F shows expression 21 days after inoculation in ICB-resistant mice. Scale bar =500 μm. Images representative of n=3 independent samples per group.

FIGS. 2A-2E show that bulk RNA sequencing of IN implant identifies differentially expressed, delta-normalized genes correlative of ICB-response. FIG. 2A is a schematic of implanting mice with IN, inoculating with 4T1, explanting IN, administering anti-PD-1, and monitoring ICB-response. FIG. 2B shows clustering of DEseq2-normalized gene expressions with principal component analysis. Clustering represents panel of 237 differentially expressed genes. FIG. 2C shows clustering of delta (D21-D7) normalized gene expressions (panel of 237 genes). FIG. 2D is a heat map of EN-identified monitoring signature of 16 differentially expressed genes. FIG. 2E shows clustering of ICB-sensitive and ICB-resistant mice based on the 16-gene panel. For FIG. 2B-2E analyses was performed on delta-normalized counts. For FIG. 2C-2E visualization was performed on delta normalized counts.

FIGS. 3A-3D show lymphocyte and myeloid cell pathways differentially regulated between ICB-sensitive and ICB-resistant mice at IN implant. Pathways associated with (FIG. 3A) general immune, (FIG. 3B) cytokine/chemokine, (FIG. 3C) myeloid cell (innate immune cell), and (FIG. 3D) lymphocyte (adaptive immune cell) responses are differentially regulated. Normalized enrichment scores (NES) represent pathways upregulated in ICB-sensitivity, cutoff of NES>1 utilized for GSEA analysis.

FIGS. 4A-4D show IN Implant captures divergent lymphocyte and myeloid cell responses as result of ICB-sensitivity versus resistance. FIG. 4A is a schematic of implanting mice with IN, inoculating with 4T1, administering anti-PD-1, and isolating PT, IN implant, and spleen for analysis with flow cytometry. FIG. 4B and FIG. 4C show flow cytometry analysis of tissues isolated on D21 post-tumor inoculation with fluorophores labelling myeloid cells (FIG. 4B) and lymphocytes (FIG. 4C). Cell proportions quantified as % of CD45+. FIG. 4D shows the ratio of myeloid cells-to-lymphocytes. Two-tailed unpaired t-tests assuming unequal variance were performed for single comparisons between two conditions. *p<0.05.

FIGS. 5A-5G show analysis of IN-derived analytes before administering therapy identifies predictive signature for ICB-response. FIG. 5A shows PCA-based clustering of DEseq 2-normalized gene expressions. Clustering represents panel of 331 differentially expressed genes. FIG. 5B is a heat map of EN-identified predictive signature of 16 genes. FIG. 5C shows clustering of mice before administering therapy based on the 16-gene predictive signature. FIGS. 5D-5G show GSEA analysis of gene expressions before therapy for (FIG. 5D) general immune, (FIG. 5E) cytokine/chemokine, (FIG. 5F) myeloid cell (innate immune cell), and (FIG. 5G) lymphocyte (adaptive immune cell) pathways. Cutoff of NES>1 used for GSEA analysis.

FIG. 6A shows longitudinal primary tumor volumes of all mice receiving anti-PD-1 versus isotype control. FIG. 6B shows longitudinal primary tumor volumes of mice administered anti-PD-1 that either did or did not receive surgical implantation of IN. Bars show mean±SEM.

FIG. 7A shows longitudinal primary tumor volumes of ICB-sensitive and ICB-resistant cohorts in bulk RNA-seq study. FIG. 7B shows longitudinal PT volumes by individual mouse from both cohorts. Bars show mean±SEM. Two-tailed unpaired t-tests assuming unequal variance were performed for single comparisons between two conditions. *p<0.05 FIGS. 8A-8D show analysis of IN-derived gene expression after therapy. FIG. 8A and

FIG. 8B show 3D clustering of differentially expressed genes after therapy. (FIG. 8A) Front face—PCA 1 vs PCA2. (FIG. 8B) Front face—PCA 3 vs PCA2. FIG. 8C and FIG. 8D show 2D clustering of differentially expressed genes after therapy. (FIG. 8C) PCA performed on all samples, whereas (FIG. 8D) PCA performed on just samples after therapy. Clustering represents panel of 237 differentially expressed genes. FIG. 8E is a heat map of EN identified gene signature of 22 genes. FIG. 8F shows clustering of the 22-gene signature. FIG. 8G shows variable importance of genes in the panel, as defined by Gini index (arbitrary units).

FIGS. 9A-9D show analysis of IN-derived gene expression with delta normalization.

FIG. 9A and FIG. 9B show 3D clustering of differentially expressed genes identified with T-tests performed on delta-normalized counts. (FIG. 9A) Front face—PCA 1 vs PCA2. (FIG. 9B) Front face—PCA 3 vs PCA2. Clustering was performed on DEseq2-normalized counts. FIG. 9C shows categorization metrics for EN-identified 16-gene signature. Sensitivity, specificity, and categorization efficiency calculated with delta normalized counts. FIG. 9D shows Clustering of delta-normalized counts during (D14) and after (D21) therapy. Delta normalization-gene expressions at D21 or D14 are normalized to gene expressions at D7.

FIGS. 10A-10E show analysis of IN-derived gene expression before therapy. FIG. 10A and FIG. 10B show 3D clustering of differentially expressed genes after therapy. (FIG. 10A) Front face—PCA 1 vs PCA2. (FIG. 10B) Front face—PCA 3 vs PCA2. FIG. 10C shows 2D clustering of differentially expressed genes before therapy. Clustering performed on DEseq2-normalized counts for 331 differentially expressed genes. FIG. 10D shows categorization metrics (sensitivity, specificity, and categorization efficiency) calculated for EN-identified predictive signature of 16 genes FIG. 10E shows relative importance of each of the 16 panel genes in distinguishing ICB sensitivity before therapy, as defined by Gini index (arbitrary units). FIGS. 11A-11C show RNAseq analysis of Cd11b+ and Cd11b− cells at day 21 after tumor inoculation with ICB treatment. The results indicate that the signature is derived from genes that are expressed by both myeloid (CD11b+) and non-myeloid (CD11b−) cells. FIG. 11A shows myeloid scores of day 21 expression for all expressed genes. FIG. 11B shows myeloid scores of day 21 expression for differentially expressed genes between Resistant and Sensitive groups across all time points. FIG. 11C shows myeloid scores of day 21 expression for 33 signature genes between Resistant and Sensitive groups across all time points.

FIGS. 12A-12D show RNAseq analysis of gene expression after therapy. FIG. 12A shows a heatmap of differentially expressed genes between sensitive and resistant groups based upon sequencing data from samples collected at days 7 (before treatment), 14 and 21 (following treatment). FIG. 12B shows clustering analysis of DEGs. FIG. 12C shows a ROC curve showing sensitivity and specificity of the gene signature. FIG. 12D shows singular value decomposition metric vs Random Forest metric scoring system for D21-D7. n=10 per group and ellipses=70% confidence intervals (arbitrary units).

FIGS. 13A-13D show categorization metrics for IN-identified 22-gene serial signature.

FIG. 13A shows receiver operating characteristic curve with sensitivity, specificity, and categorization efficiency between ICB-sensitive and ICB-resistant groups calculated for serial-normalized gene expression during therapy (D14-D7). FIG. 13B shows receiver operating characteristic curve for after therapy serial analysis (D21-D7) with sensitivity, specificity, and categorization efficiency between ICB-sensitive and ICB-resistant groups calculated for serial-normalized gene expression; red dotted line represents 50% arca under the curve. FIG. 13C shows scoring principal component analysis clustering for serial-normalized counts after (D21-D7) therapy by ICB response, ****p<0.01. FIG. 13D shows relative importance of each of the 22 panel genes in SVD-RF scoring, as defined by Gini index (arbitrary units).

FIG. 14 shows validation for the 16-gene before ICB signature with or without sample partitioning for cross validation compared to the Oncogene DX® panel represented by \hat{ } and panels associated with TNBC responsiveness to ICB from Comput Biol Med. 2023; 161: 10706 and Breast Cancer. 2022; 29(4): 666-676 represented by * and +, respectively.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), clement(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). For example, “treating” cancer may refer to reducing the size of a tumor, reducing the number of tumors, eliminating a tumor, reducing the risk of metastasis of a tumor, and the like. As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

The terms “subject” and “patient” are used interchangeably herein and refer to any animal. In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

### DETAILED DESCRIPTION

Immunotherapy has emerged as a promising treatment for cancer, with more than 60% of these therapeutic approaches targeting T cells. T cells are powerful mediators of anti-tumor immune surveillance through their ability to detect and eliminate cancerous cells. Immune checkpoint blockade (ICB) modulating T cell regulation has become the most successful immunotherapy in a variety of tumors, including the highly immunogenic triple negative breast cancer (TNBC). Pembrolizumab, an antibody to programmed cell death protein 1 (anti-PD-1), has been approved for treating early, locally advanced, and metastatic TNBC. These therapies aim to block PD-L1 signaling from pro-tumor cells, including monocytes, macrophages, fibroblasts, and tumor cells, through binding to the PD-1 receptor on T cells. Nevertheless, only 10-20% of PD-L1+ metastatic TNBC patients who meet selection criteria benefit from ICB.

Accordingly, biomarkers to stratify patients by likelihood of response are needed for effective treatment outcomes.

A widely used biomarker for stratifying sensitivity to anti-PD-1 is PD-L1 expression on tumor infiltrating lymphocytes or the tumor stroma. Surprisingly, some PD-L1 negative patients are sensitive to ICB, demonstrating the limitations of PD-L1 alone to stratify patients. Tumor mutational burden (TMB) has also been utilized as an ICB biomarker in solid tumors, yet the majority of even these biomarker-positive patients are ICB-resistant. Additionally, tumor-derived gene signatures have been pursued for predicting ICB response, but similarly fail to effectively stratify patients with TNBC. In sum, PD-L1 status, TMB, and tumor-derived gene signatures do not accurately predict a clinical response to ICB. Accordingly, there remains an unmet need for accurately stratifying TNBC patients by likelihood of response to immunotherapy. The present disclosure addresses this need and provides a method that can be used to accurately stratify patients based upon likelihood of response to immunotherapy.

In some embodiments the present disclosure provides a method involving subcutaneously implanting a microporous scaffold in a subject, with the pores supporting infiltration of host cells and vascularization. Immune cells within the vasculature are recruited to this environment due to the foreign body response, and thus the local microenvironment is dynamically modified through disease initiation and progression as an immunologic niche (IN). Dynamic gene expression within the IN was investigated herein to provide biomarkers that correlate with the response to anti-PD-1 in TNBC. The 4T1 model of metastatic TNBC was used, which was treated with anti-PD-1 and resulted in cohorts that were either sensitive or resistant indicated by the growth of the primary tumor and survival. The IN was sampled during and after ICB and sequenced to identify gene expression signatures that correlated with sensitivity or resistance. Gene expression was also analyzed at the IN prior to ICB treatment to identify markers predicting therapeutic response. Longitudinally interrogating an IN, to monitor changes associated with ICB response, presents a new opportunity to personalize care and investigate mechanisms underlying treatment resistance.

In some aspects, provided herein are methods. In some embodiments, the methods comprise obtaining a sample from a microenvironment of a synthetic scaffold implanted in a subject, and measuring an expression level or an amount of at least one RNA, at least one gene, at least one cell type, and/or at least one protein in the sample. In some embodiments, the methods comprise obtaining a sample from a microenvironment of a synthetic scaffold implanted in a subject, and measuring an expression level or an amount of a panel of RNAs, a panel of genes, at least one cell type, and/or a panel of proteins in the sample. As used herein, the term “panel” indicates two or more (e.g. two or more RNAs, genes, or proteins).

In some embodiments, the methods comprise measuring expression level of nucleic acid (e.g. DNA, RNA) and/or protein in a sample. In some embodiments, the methods comprise measuring expression level of one or more genes in a sample. In some embodiments, the methods comprise measuring expression level of a panel of genes in the sample. In some embodiments, the panel of genes comprises at least 2, at least 3, at least 4, at least 5, at least 6, at least 7, at least 8, at least 9, at least 10, at least 11, at least 12, at least 13, at least 14, at least 15, or at least 16 genes. In some embodiments, the panel of genes comprises less than 1000 genes.

In some embodiments, the panel of genes comprises less than 500 genes. In some embodiments, the panel of genes comprises less than 100 genes. In some embodiments, the panel of genes comprises between 2 and 100 genes. For example, in some embodiments the panel of genes comprises at least 10 but less than 100 genes. In some embodiments, the methods may comprise measuring a level of RNA (e.g. mRNA) encoding a gene. In some embodiments, the methods comprise measuring expression level of one or more proteins in a sample. In some embodiments, the methods comprise analyzing cells from the sample. For example, analyzing cells may comprise assessing cell types (e.g. cell sub-populations) present within the sample. In some embodiments, analyzing cells comprises measuring an amount of one or more cell types in a sample. In some embodiments, analyzing cells comprises measuring an amount of two or more cell types in a sample and calculating a ratio of one cell type to another cell type in the sample.

In some embodiments, the sample is obtained from a scaffold implanted in the subject. In some embodiments, the subject is diagnosed with or at risk of having cancer. The cancer may be any type of cancer. In some embodiments, the subject is diagnosed with or at risk of having a cancer selected from breast cancer, bladder cancer, cervical cancer. colon cancer, head and neck cancer, Hodgkin lymphoma, liver cancer, lung cancer, kidney cancer (e.g. renal cell cancer) skin cancer (e.g. melanoma), stomach cancer, or rectal cancer. In some embodiments, the subject is diagnosed with or at risk of having breast cancer. In some embodiments, the breast cancer is triple negative breast cancer (e.g. breast cancer wherein cancer cells do not have estrogen receptors, progesterone receptors, and do not produce substantial amounts of the protein HER2).

In some embodiments, the scaffold is implanted in the subject after the subject is diagnosed with cancer. In some embodiments, the subject diagnosed with cancer has undergone surgery (e.g. surgical resection) to remove a tumor and is being monitored for or at risk of tumor recurrence. In some embodiments, the scaffold is implanted in the subject after the subject diagnosed with cancer or at risk of having cancer has received a treatment with an immune checkpoint inhibitor. In some embodiments, the subject has undergone surgery to remove the tumor and has received treatment with an immune checkpoint inhibitor. In some embodiments, the subject has not received surgery and has received treatment with an immune checkpoint inhibitor. In some embodiments, the sample is obtained from the microenvironment of the synthetic scaffold about 1 day to about 28 days after the subject has received treatment with the immune checkpoint inhibitor. In some embodiments the sample is obtained from the microenvironment of the synthetic scaffold about 7 days to about 21 days after the subject has received treatment with the immune checkpoint inhibitor. In some embodiments, the sample is obtained from the microenvironment of the synthetic scaffold about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days after the subject has received treatment with the immune checkpoint inhibitor.

In some embodiments, the methods comprise monitoring response to the immune checkpoint inhibitor in the subject. For example, in some embodiments the methods of monitoring response to the immune checkpoint inhibitor comprise determining whether the subject is sensitive or resistant to treatment with the immune checkpoint inhibitor based upon the expression level, presence of, or amount of the at least one RNA, at least one gene, at least one cell type, and/or at least one protein in the sample. As used herein, the term “sensitive” indicates a positive response to the immune checkpoint inhibitor. Accordingly, the term “sensitive” indicates that the immune checkpoint inhibitor is effective at treating the cancer (e.g. reducing the tumor size, inhibiting tumor growth, reducing the number of tumors, reducing the risk of metastasis, etc.). In contrast, the term “resistant” indicates a lack of a positive response to the immune checkpoint inhibitor. Accordingly, the term “resistant” indicates that the immune checkpoint inhibitor is not effective at treating the cancer.

In some embodiments, the scaffold is implanted in a subject diagnosed with or at risk of having cancer that has not received treatment with an immune checkpoint inhibitor. In some embodiments, the subject has undergone surgery (e.g. surgical resection) to remove a tumor and has not received treatment with an immune checkpoint inhibitor. In some embodiments, the methods comprise predicting response to an immune checkpoint inhibitor in the subject. For example, in some embodiments the methods comprise predicting whether the subject is likely to be sensitive to or is likely to be resistant to treatment with an immune checkpoint inhibitor based upon the expression level or amount of the at least one RNA, at least one gene, at least one cell type, and/or at least one protein in the sample.

For any of the embodiments and methods described herein, the immune checkpoint inhibitor may be any suitable immune checkpoint inhibitor, including agents that target checkpoint proteins including PD-1, PD-L1, B7-1/B7-2, LAG-3, and CTLA-4. In some embodiments, the immune checkpoint inhibitor is a monoclonal antibody. For example, the immune checkpoint inhibitor may be an antibody that binds PD-1, PD-L1, B7-1/B7-2, LAG-3, or CTLA-4. Exemplary immune checkpoint inhibitors include, for example PD-1 inhibitors (e.g. pembrolizumab, nivolumab, cemiplimab), PD-L1 inhibitors (e.g. atezolizumab, avelumab, durvalumab), CTLA-4 inhibitors (ipilimumab), LAG-3 inhibitors (relatlimab), or combinations thereof.

The terms “scaffold”, “biomaterial scaffold, and “synthetic scaffold” are used interchangeably herein and refer to any scaffold which is implanted in the subject and subsequently used to collect a sample from the subject. Suitable scaffolds are described in U.S. Patent Publication No. 2020/0323893A1 and U.S. Patent Publication No. 2021/0382050, the entire contents of which are incorporated herein by reference.

In some embodiments, the scaffold is porous and/or permeable. In some embodiments, the scaffold comprises a polymeric matrix. In some embodiments, the scaffold acts as a substrate permissible for inflammation due to, for example, cancer. In some embodiments, the scaffold provides an environment for attachment, incorporation, adhesion, encapsulation, etc. of agents (e.g., DNA, lentivirus, protein, cells, etc.) that create a capture site within the scaffold. In some embodiments, agents are released (e.g., controlled or sustained release) to attract circulating cells or molecules indicative of cancer status.

In some embodiments, the scaffold or a portion thereof is configured for sustained release of agents. In some embodiments, the sustained release provides release of biologically active amounts of the agent over a period of at least 30 days (e.g., 40 days, 50 days, 60 days, 70 days, 80 days, 90 days, 100 days, 180 days, etc.).

In some embodiments, the scaffold is partially or exclusively composed of a micro-porous poly(lactide-co-glycolide) (PLG) biomaterial. In some embodiments, the scaffold is partially or exclusively composed of a micro-porous poly(e-caprolactone) (PCL), forming a PCL scaffold. Such PCL scaffolds may have a greater stability than the micro-porous poly(lactide-co-glycolide) (PLG) biomaterial scaffolds. In exemplary embodiments, the scaffold comprises PCL and/or PEG and/or alginate and/or PLG. In some embodiments, the scaffold is formed partially or exclusively of hydrogel. For example, the scaffold may be formed partially or exclusively of hydrogel, e.g., a poly(ethylene glycol) (PEG) hydrogel, to form a PEG scaffold. In some embodiments, the scaffold is a controlled release PEG scaffold. Any PEG is contemplated for use in the compositions and methods of the disclosure. In general, the PEG has an average molecular weight of at least about 5,000 daltons. In some embodiments, the PEG has an average molecular weight of at least 10,000 daltons. In some embodiments, the PEG has an average molecular weight of at least 15,000 daltons. In some embodiments, the PEG has an average molecular weight between 5,000 and 20,000 daltons, or between 15,000 and 20,000 daltons. In some embodiments, the PEG has an average molecular weight of 5,000, of 6,000, of 7,000, of 8,000, of 9,000, of 10,000, of 11,000, of 12,000 of 13,000, of 14,000, of 15,000, of 16,000, or 17,000, or 18,000, or 19,000, of 20,000, of 21,000, of 22,000, of 23,000, or 24,000, or of 25,000 daltons. In some embodiments, the PEG is a four-arm PEG. In some embodiments, each arm of the four-arm PEG is terminated in an acrylate, a vinyl sulfone, or a maleimide. It is contemplated that use of vinyl sulfone or malcimide in the PEG scaffold renders the scaffold resistant to degradation. It is further contemplated that use of acrylate in the PEG scaffold renders the scaffold susceptible to degradation.

In some embodiments, one or more agents are associated with a scaffold. For example, agents may be associated with the scaffold to establish a hospitable environment for markers of cancer or a response to an anti-cancer treatment, such as an immune checkpoint inhibitor. As another example, one or more agents may be associated with a scaffold to provide a therapeutic benefit to a subject. Agents may be associated with the scaffold by covalent or non-covalent interactions, adhesion, encapsulation, etc. In some embodiments, a scaffold comprises one or more agents adhered to, adsorbed on, encapsulated within, and/or contained throughout the scaffold. The present invention is not limited by the nature of the agents. Such agents include, but are not limited to, peptides, proteins, nucleic acid molecules, small molecule drugs, lipids, carbohydrates, cells, cell components, and the like. In various embodiments, the agent is a therapeutic agent. In some embodiments, two or more (e.g., 3, 4, 5, 6, 7, 8, 9, 10 . . . 20 . . . 30 . . . 40 . . . , 50, amounts therein, or more) different agents are included on or within the scaffold. In other embodiments, no agents are provided with the scaffold. In some aspects, the scaffold is modified to deliver proteins, peptides, small molecules, gene therapies, biologics, etc.

In some embodiments, the scaffold comprises a polymeric matrix. In some embodiments, the matrix is prepared by a gas foaming/particulate leaching procedure, and includes a wet granulation step prior to gas foaming that allows for a homogeneous mixture of porogen and polymer and for sculpting the scaffold into the desired shape. In some embodiments, scaffolds may be formed of a biodegradable polymer, e.g., PCL, that is fabricated by emulsifying and homogenizing a solution of polymer to create microspheres. Other methods of microsphere production are known in the art and are contemplated by the present disclosure. See, e.g., U.S.

Patent Application Publication Numbers 2015/0190485 and 2015/0283218, each of which is incorporated herein in its entirety. The microspheres are then collected and mixed with a porogen (e.g., salt particles), and the mixture is then pressed under pressure. The resulting discs are heated, optionally followed by gas foaming. Finally, the salt particles are removed. The fabrication provides a mechanically stable scaffold which does not compress or collapse after in vivo implantation.

Scaffolds of the present disclosure may comprise any of a large variety of structures including, but not limited to, particles, beads, polymers, surfaces, implants, matrices, etc. Scaffolds may be of any suitable shape, for example, spherical, generally spherical (e.g., all dimensions within 25% of spherical), ellipsoidal, rod-shaped, globular, polyhedral, etc. The scaffold may also be of an irregular or branched shape.

In some embodiments, a scaffold comprises nanoparticles or microparticles (e.g., compressed or otherwise fashioned into a scaffold). In various embodiments, the largest cross-sectional diameters of a particle within a scaffold is less than about 1,000 μm, 500 μm, 200 μm, 100 μm, 50 μm, 20 μm, 10 μm, 5 μm, 2 μm, 1 μm, 500 nm, 400 nm, 300 nm, 200 nm or 100 nm. In some embodiments, a population of particles has an average diameter of: 200-1000 nm, 300-900 nm, 400-800 nm, 500-700 nm, etc. In some embodiments, the overall weights of the particles are less than about 10,000 kDa, less than about 5,000 kDa, or less than about 1,000 kDa, 500 kDa, 400 kDa, 300 kDa, 200 kDa, 100 kDa, 50 kDa, 20 kDa, 10 kDa.

In some embodiments, a scaffold comprises PCL. In further embodiments, a scaffold comprises PEG. In certain embodiments, PCL and/or PEG polymers and/or alginate polymers are terminated by a functional group of chemical moiety (e.g., ester-terminated, acid-terminated, etc.).

In some embodiments, the charge of a matrix material (e.g., positive, negative, neutral) is selected to impart application-specific benefits (e.g., physiological compatibility, beneficial interactions with chemical and/or biological agents, etc.). In certain embodiments scaffolds are capable of being conjugated, either directly or indirectly, to a chemical or biological agent). In some instances, a carrier has multiple binding sites (e.g., 2, 3, 4, 5, 6, 7, 8, 9, 10 . . . 20 . . . 50 . . . 100, 200, 500, 1000, 2000, 5000, 10,000, or more).

In some embodiments, the lifetimes of the scaffolds are well within the timeframe of clinical significance are demonstrated. For example, stability lifetimes of greater than 90 days are contemplated, with percent degradation profiles of less than about 50%, 45%, 40%, 35%, 30%, 25%, 20%, 15%, 10%, 5%, and 1% respectively, where the percent degradation refers to the scaffolds' ability to maintain its structure for sufficient cell capture as a comparison of its maximum capture ability. Such ability is measured, for example, as the change in porous scaffold volume over time, the change in scaffold mass over time, and/or the change in scaffold polymer molecular weight over time. These long lifetimes mean that scaffolds can now be applied in patient-friendly conditions that allow subjects to wear the scaffold under normal daily living conditions, inside and outside the clinical environment.

In some embodiments, the scaffold or a portion thereof is configured to be sufficiently porous to permit cells and molecules of interest into the pores. The size of the pores may be selected for particular cell types of interest and/or for the amount of ingrowth desired and are, for example without limitation, at least about 20 μm, 30 μm, 40 μm, 50 μm, 100 μm, 200 μm, 500 μm, 700 μm, or 1000 μm. In some embodiments, scaffold is not porous but is instead characterized by a mesh size that is, e.g., 10 nanometers (nm), 15 nm, 20 nm, 25 nm, 30 nm, 40 nm, or 50 nm.

The scaffold may be implanted at any suitable location in the body of the subject. For example, the scaffold may be implanted subcutaneously. As another example, the scaffold may be implanted in a fat pad. In some embodiments, the scaffold is implanted proximal to the site of a tumor or suspected tumor in the subject. In some embodiments, the scaffold is implanted at a separate site, away from the site of the actual or suspected tumor. In some embodiments, more than one scaffold is implanted in the subject. For example, at least 1, at least 2, at least 3, at least 4, at least 5, at least 6, at least 7, at least 8, at least 9, or at least 10 scaffolds may be implanted in a subject. In some embodiments, samples from each of the scaffolds implanted in the subject are obtained and biomarker (e.g., expression) profiles of each sample are measured.

Implantation of synthetic scaffolds in a subject (e.g. in the subcutaneous space or fat pad of a subject), trigger in vivo events (e.g., a foreign body response, an immune response against the scaffold) that result in the creation of a niche in the subject at the implantation site. In some embodiments, the sample is obtained from the niche. As used herein, the term “niche” refers to the area of cells and molecules located at or near the site at which a synthetic scaffold is or was implanted in the subject (e.g. at the scaffold implantation site). In some embodiments, the niche reflects the in vivo events caused by the implantation of the scaffold. In some aspects, the niche is physically attached to the scaffold. In some embodiments, the niche comprises cells and other molecules or factors involved in an immune response against the synthetic scaffold implanted in the subject at the implantation site. In some embodiments, the niche may be representative of the patient's health status. For example, the niche may be representative of the patient's status with regard to cancer or a treatment for cancer. For example, the niche may be representative of whether a patient is afflicted with cancer, whether a subject is likely to be sensitive to an anti-cancer treatment (e.g. an immune checkpoint inhibitor), whether a subject is likely to be resistant to an anti-cancer treatment (e.g. an immune checkpoint inhibitor), whether a subject is exhibiting a positive or a negative response to an anti-cancer treatment (e.g. whether the patient is sensitive to an immune checkpoint inhibitor or resistant to an immune checkpoint inhibitor), and the like. In some embodiments, the content of the niche changes as the health status (e.g. cancer, response to treatment with an anti-cancer treatment) changes. In some embodiments, the sample is obtained from the niche. For example, in some embodiments the niche is biopsied, and the analysis of the biopsied sample allows for the disease diagnosis and/or prognosis, in addition to treatment selection and monitoring.

In some embodiments, the methods described herein involve measuring an expression level or an amount of at least one gene, RNA, cell type, and/or protein in a sample. In some embodiments, the methods comprise analyzing cells from the sample. For example, in some embodiments the methods involve assessing cell types (e.g. cell sub-populations) present within the sample. For example, in some embodiments the methods comprise measuring an amount of at least one cell type present within the sample. In some embodiments, the methods comprise measuring an amount of two or more cell types present within the sample. In some embodiments, the methods comprise measuring an amount of a first cell type and a second cell type present within the sample and calculating a ratio of the amount of the first cell type: the amount of the second cell type in the sample. Analyzing cells may be performed as an alternative to or in addition to measuring expression of a gene, RNA, or protein in the sample.

In some embodiments, the sample is obtained from the scaffold microenvironment. The term “microenvironment” when used in reference to the scaffold (e.g. “scaffold microenvironment”, “microenvironment of the scaffold”, etc.) is used herein to include the scaffold, a portion of the scaffold, and the niche. In some embodiments, the sample is obtained from the scaffold or a portion thereof. In some embodiments, the sample is obtained from the niche. The sample obtained from the scaffold microenvironment may be isolated from the subject prior to use for analysis of an expression level or amount of a gene, RNA, cell type, or protein in the sample.

In some embodiments, the scaffold or a portion thereof is retrieved from the subject to provide the sample from which expression is measured. For example, the scaffold or a portion thereof may be biopsied, and used to obtain the sample from which DNA/RNA/protein expression is measured and/or cells are analyzed. In some embodiments, the methods involve measuring a level of expression of a gene, an RNA, e.g., a messenger RNA (mRNA), or a protein, in a sample obtained from a niche at the implantation site of a synthetic scaffold. In some embodiments, the methods involve analyzing cells in a sample (e.g. determining populations of cell types present within a sample) obtained from a niche at the implantation site of a synthetic scaffold. In some embodiments, the methods comprise measuring an expression level of at least one gene and analyzing cells in the sample. For example, in some embodiments the methods comprise measuring an expression level of at least one gene and determining an amount of at least one cell type in the sample. In some embodiments the methods comprise measuring an expression level of at least two genes and determining an amount of at least two cell types in the sample. For example, at a time point of interest a biopsy may be performed to remove the scaffold or a portion thereof. For example, a biopsy may be performed to remove the entire scaffold. Alternatively, a core-needle biopsy may be performed to remove a portion of the scaffold microenvironment (e.g. a portion of the scaffold or a portion of the niche). With the scaffold or a portion thereof removed, or a sample of the niche of the scaffold removed, the contents may be processed for molecular content determination. In some examples, such processing includes isolating RNA and/or protein (e.g., by homogenization in Trizol reagent or detergent, respectively).

In some embodiments, the contents of the sample are processed to analyze cells contained within the sample. For example, cell populations may be assessed by techniques including fluorescent-assisted cell sorting (FACS), magnetic-assisted cell sorting, and/or histological techniques including immunohistochemistry or fluorescent imaging technologies. Optionally, in some examples, cell populations within the scaffold may be fractionated. For example, cell populations may be fractionated for cell type specific analysis. Suitable fractionation techniques include methods that separate cell populations based on fluorescent-assisted cell sorting or magnetic-assisted cell sorting. In some embodiments, an amount of at least one cell type in the sample is determined. In some embodiments, an amount of at least two cell types in the sample is determined. In some embodiments, a ratio of an amount of a first cell type: an amount of a second cell type in the sample is determined.

In some embodiments, RNA and/or protein is isolated from the scaffold or portion thereof or from the niche, and used for analysis of gene or protein expression. Analysis of gene or protein expression may be achieved, in some examples, using either qRT-PCR or RNAseq, for gene expression, and cither ELISA or Luminex (bead-based multiplex assays), for protein expression.

In some embodiments, the methods comprise measuring a combination of at least two of an expression level of a gene, an RNA, and a protein. In some embodiments, the methods comprise measuring the expression level of at least one gene, at least one RNA, and at least one protein. In some embodiments, the methods comprise measuring the expression level of a plurality of different genes, a plurality of RNA, and/or a plurality of proteins. In some embodiments, the methods comprise measuring the expression level of at least 2, 3, 4, 5 or more genes, at least 2, 3, 4, 5 or more RNA, and/or at least 2, 3, 4, 5 or more proteins in the sample. In some embodiments, the methods comprise measuring the expression level of at least 10, 15, 20 or more genes, at least 10, 15, 20 or more RNA, and/or at least 10, 15, 20 or more proteins in the sample. In some embodiments, the methods comprise measuring the expression level of at least 50,100, 200 or more genes, at least 50, 100, 200 or more RNA, and/or at least 50, 100, 200 or more proteins in the sample.

In some embodiments, the methods comprise measuring the expression level of at least 2 different genes. In some embodiments, the methods comprise measuring the expression level of at least 2, at least 4, at least 6, at least 8, at least 10, at least 12, at least 14, or at least 16 different genes. In some embodiments, the methods comprise measuring the expression level of more than 10 different genes, more than 100 different genes, more than 1000 different genes, or more than 5000-10,000 different genes, and the expression levels of the different genes constitute a gene signature.

In some embodiments, the methods comprise measuring the expression level of more than 10 different RNA, more than 100 different RNA, more than 1000 different RNA, more than 5000-10,000 different RNA, and the expression levels of the different RNA constitute an RNA signature, or a transcriptome.

In some embodiments, the methods comprise measuring an expression level or amount of a gene, an RNA, a cell type, or a protein indicative of one or more regulatory pathways involved in cancer, including cytokine signaling, chemokine regulation, leukocyte proliferation, leukocyte migration, leukocyte differentiation, leukocyte cell-cell adhesion, leukocyte chemotaxis, inflammatory responses, leukocyte cytotoxicity, and aberrant inflammation.

In some embodiments, the method comprises measuring expression level of one or more genes selected from Prostate Transmembrane Protein, Androgen Induced 1 (Pmepa1), predicted gene, 50439 (Gm50439), TNF Superfamily Member 9 (Tnfsf9), GAR1 Ribonucleoprotein (Gar1), Forkhead Box F2 (Foxf2), Cytokine Receptor Like Factor 1 (Crlf1), predicted gene 11847 (Gm11847), Inhibitor of DNA Binding 3, HLH Protein (Id3), Aldehyde Dehydrogenase 1 Family Member A2 (Aldh1a2), Solute Carrier Family 5 Member 7 (Slc5a7), predicted gene 49484 (Gm49484), R-Spondin 2 (Rspo2), Histone Deacetylase 1 (Hdac1), X2200002j24Rik, Sh3gf3, and Slc2a12. In some embodiments, the method comprises measuring the expression level of at least 2, at least 3, at least 4, at least 5, at least 6, at least 7, at least 8, at least 9, at least 10, at least 11, at least 12, at least 13, at least 14, or at least 15 genes selected from Pmepa 1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the method comprises measuring expression level of each of Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, SH3 Domain Containing GRB2 Like 3, Endophilin A3 (Sh3gf3), and Solute Carrier Family 2 Member 12(Slc2a12).

In some embodiments, the method comprises measuring expression level of one or more genes selected from Heparan Sulfate 6-O-Sulfotransferase 2 (Hs6st2), predicted gene 48732 (Gm48732), predicted gene 50393 (Gm50393), Guanylate Binding Protein 2 (Gbp2), Receptor Transporter Protein 4 (Rtp4), Guanylate Binding Protein 3 (Gbp3), Tryptase Alpha/Beta 1 (Tpsab1), SURF1 Cytochrome C Oxidase Assembly Factor (Surf1), Ring Finger Protein 144A (Rnf144a), Stefin A2 (Stfa2), Cytokine Receptor Like Factor 1 (Crlf1), SprT-Like N-Terminal Domain (Sprtn), SH3 Domain Containing GRB2 Like 3, Endophilin A3 (Sh3g13), Nucleoporin like 1 (Nupl1), Tlr12, and Alms1. In some embodiments, the method comprises measuring the expression level of at least 2, at least 3, at least 4, at least 5, at least 6, at least 7, at least 8, at least 9, at least 10, at least 11, at least 12, at least 13, at least 14, or at least 15 genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Toll-like receptor 12 (Tlr12), and ALMS1 centrosome and basal body associated protein (Alms1). In some embodiments, the method comprises measuring expression level of each of Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1.

In some embodiments, the method comprises measuring the expression level of one or more genes selected from Solute Carrier Family 7 Member 14 (Slc7a14), Dickkopf WNT Signaling Pathway Inhibitor 2 (Dkk2), Farnesyl Diphosphate Synthase (Fdps), Tribbles Pseudokinase 1 (Trib1), PAXIP1 Associated Glutamate Rich Protein 1A (Pagrla), Colony Stimulating Factor 1 (Csf1), Prostaglandin-Endoperoxide Synthase 2 (Ptgs2), Gm50393, Murine MHC class Ib gene (H2-M2), Gm19195, Synaptonemal Complex Protein 2 (Sycp2), X2810457G06Rik, Gm48996, Solute Carrier Family 4 Member 4 (Slc4a4), D-Aminoacyl-TRNA Deacylase 2 (Dtd2), Laeverin (Lvrn), Ring Finger Protein 144A (Rnf144a), Zic Family Member 4 (Zic4), nucleoporin 58 (Nupl1), Cd177 molecule (Cd177), Stefin-2 (Stfa2), and Ripply Transcriptional Repressor 3 (Ripply3). In some embodiments, the method comprises measuring the expression level of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes selected from Slc7a14, Dkk 2, Fdps, Trib 1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the method comprises measuring expression level of each of Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3.

In some embodiments, the method comprises measuring the expression level or one or more genes selected from Chondroitin Sulfate Synthase 1(Chsy1), SprT-Like N-Terminal Domain (Sprtn), Gm18935, Translocase Of Inner Mitochondrial Membrane 44 (Timm44), Gm19056, Rp118.ps2, toll-like receptor 12 (Tlr12), X2900078111Rik, Cytokine Receptor Like Factor 1 (Crlf1), Cilia And Flagella Associated Protein 251 (Wdr66), Phospholipase A2 Group XIIA (Pla2g12a), Gm9118, Synapse Defective Rho GTPase Homolog 2 (Syde2), A530010L16Rik, Gm183562, LIM Homeobox (Lhx6), Testis Expressed 22 (Tex22), Glutamate Receptor Interacting Protein 1 (Grip1), X8430429K09Rik, Gm6361, and Prostaglandin-Endoperoxide Synthase 2 (Ptgs2).). In some embodiments, the method comprises measuring the expression level of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes selected from Chsy 1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, the method comprises measuring the expression level of each of Chsy1, Sprtn, Gm18935, Timm44, Gm19056, Rp118.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2.

In some embodiments, the methods described herein comprise comparing the expression level (e.g. of the gene, RNA, or protein) in the sample or the amount of a given cell type in the sample to a control level or a cutoff level (e.g. a threshold level). For example, the expression level in the sample may be compared to a control level. In some aspects, the control level is that of a control subject which may be a matched control (e.g. a control of the same species, gender, ethnicity, age group, smoking status, BMI, current therapeutic regimen status, medical history, or a combination thereof as the subject), but differs from the subject being diagnosed in that the control is not afflicted with cancer. In some embodiments, the control level is an expression level obtained from the subject prior to receiving a cancer treatment (e.g. prior to receiving an immune checkpoint inhibitor). Such a control level obtained from the subject prior to receiving treatment is also referred to herein as a “baseline” level. In some embodiments, the “baseline level” is measured in a sample obtained from the scaffold microenvironment of the subject prior to the subject receiving the treatment (e.g. prior to receiving the immune checkpoint inhibitor).

In some embodiments, the expression level in the sample is compared to a cutoff level. In some embodiments, the expression level in the sample is compared to a baseline level. This can be referred to as being “normalized” to a baseline level.

In some embodiments, the expression level or amount in the sample may be increased (e.g. relative to a control level or a cutoff level). As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

In some embodiments, the expression level or amount in the sample may be decreased (e.g. relative to a control level or a cutoff level). As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

In some embodiments, increased expression level of one or more genes, RNAs, or proteins in the sample is indicative that the subject is likely to be sensitive to treatment with an immune checkpoint inhibitor. In some embodiments, increased expression level of one or more genes, RNAs, or proteins in the sample is indicative that the subject is likely to be resistant to treatment with an immune checkpoint inhibitor. In some embodiments, decreased expression level of one or more genes, RNAs, or proteins in the sample is indicative that the subject is likely to be sensitive to treatment with an immune checkpoint inhibitor. In some embodiments, decreased expression level of one or more genes, RNAs, or proteins in the sample is indicative that the subject is likely to be resistant to treatment with an immune checkpoint inhibitor.

In some embodiments, increased expression level of one or more genes, RNAs, or proteins in the sample is indicative that the subject undergoing treatment with an immune checkpoint inhibitor is sensitive to the treatment (e.g. is exhibiting a positive response to the immune checkpoint inhibitor). In some embodiments, increased expression level of one or more genes, RNAs, or proteins in the sample is indicative that the subject undergoing treatment with an immune checkpoint inhibitor is resistant to the treatment (e.g. is not exhibiting a positive response to the immune checkpoint inhibitor). In some embodiments, decreased expression level of one or more genes, RNAs, or proteins in the sample is indicative that the subject undergoing treatment with an immune checkpoint inhibitor is sensitive to the treatment (e.g. is exhibiting a positive response to the immune checkpoint inhibitor). In some embodiments, decreased expression level of one or more genes, RNAs, or proteins in the sample is indicative that the subject undergoing treatment with an immune checkpoint inhibitor is resistant to the treatment (e.g. is not exhibiting a positive response to the immune checkpoint inhibitor). The change relative to a control, threshold, or cutoff level and its significance for responsiveness or likely responsiveness to an immune checkpoint inhibitor may vary depending on the specific gene, protein, or RNA measured.

In some embodiments, the method comprises measuring the level of expression of a gene, RNA, or protein at a first time point and at a second time point, and the measured level of the first time point serves as a control level or establishes a baseline. In some embodiments, the first time point is prior to treatment (e.g. treatment with an immune checkpoint inhibitor) and the second time point is after treatment.

In some embodiments, the levels of expression are measured and the measured levels are normalized or calibrated to a level of a housekeeping gene. Any suitable housekeeping gene may be used. In some embodiments, the housekeeping gene is one or more of GAPDH, Hmbs, Tbp, Ubc, Ywhaz, Polr2a. Suitable housekeeping genes include, for example, beta-actin (ACTB), aldolase A, fructose-bisphosphate (ALDOA), glyceraldehyde-3-phosphate dehydrogenase (GAPDH), phosphoglycerate kinase 1 (PGK1), RNA polymerase II subunit A (POLR2A), lactate dehydrogenase A (LDHA), ribosomal protein S27a (RPS27A), ribosomal protein L19 (RPL19), ribosomal protein L11 (RPL11), non-POU domain containing, octamer-binding (NONO), Rho GDP dissociation inhibitor alpha (ARHGDIA), ribosomal protein L32 (RPL32), ubiquitin C (UBC), HMBS, RBP, and Ywhaz.

In some embodiments, expression levels may be measured at multiple time points. For example, a sample may be obtained from the subject (e.g. from the scaffold or a portion thereof, or from the niche) at a first time point, a second time point, a third time point, etc. In some embodiments, at least two time points are following treatment. Accordingly, the methods described herein may be used to monitor a subject across time for responsiveness to a cancer treatment such as an immune checkpoint inhibitor.

In some embodiments, the expression levels of the genes, RNA, and/or proteins are processed through an algorithm to obtain a single metric or single score of gene expression, RNA expression, or protein expression. In some embodiments, the expression levels are normalized to housekeeping gene expression levels. In some embodiments, the expression levels are processed through singular value decomposition, dynamic mode decomposition, principle component analysis, fisher linear discriminant, or linear combination. In some embodiments, the expression levels of the genes, RNA, and/or proteins (optionally normalized to housekeeping gene expression levels) or the single metric or single score is processed through a machine learning algorithm to obtain a score. For example, the score may be indicative of the likelihood of or actual sensitivity to treatment with an immune checkpoint inhibitor. In some embodiments, the metric of gene expression, RNA expression, or protein expression is combined with the prediction score to obtain a graphical or numerical output, which may be used as a control (or a panel of controls) against which the measured levels are compared.

In some embodiments, more than one sample is obtained from the scaffold, portion thereof, or niche, and each sample is obtained at a different point in time. For example, in some embodiments, 2, 3, 4, 5, 6, 7, 8, 9, 10, or more samples are obtained, each sample obtained at a different point in time. In some embodiments, a sample is obtained once a day, 2× per day, 3× per day, 4× per day or more frequently. In some embodiments, a sample is obtained every 2, 3, 4, 5, or 6 days. In some embodiments, a sample is obtained once a week. In some embodiments, a sample is obtained once every 2 weeks, 3 weeks, 4 weeks, 5 weeks, 6 weeks, 7 weeks, or 8 weeks or less frequently. In some embodiments, a sample is obtained on a regular basis based on the analysis of a first sample.

In some embodiments, at least one sample is obtained from the subject (e.g. from the scaffold, from a portion of the scaffold, or from the niche) one day or more following treatment with an immune checkpoint inhibitor. For example, in some embodiments at least one sample is obtained about 24 hours, about 36 hours, about 48 hours, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or more than 3 weeks following treatment (e.g. about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, etc.) after treatment with an immune checkpoint inhibitor.

In some embodiments, at least one sample is obtained at a first time point, and at least one sample is obtained at a second time point that is after the first time point. In some embodiments, the methods comprise determining the likelihood of sensitivity to or the actual sensitivity to treatment with an immune checkpoint inhibitor based upon whether the expression level of one or more genes, RNA, or protein is changed from the first time point to the second time point (e.g. increases or decreases).

Any suitable method may be used to determine expression levels. Suitable methods of determining expression levels of nucleic acids (e.g., mRNA) are known in the art and include amplification based techniques, such as quantitative polymerase chain reaction (qPCR), quantitative real-time PCR (qRT-PCR), and isothermal amplification methods (e.g. nicking endonuclease amplification reaction, transcription mediated amplification, loop-mediated isothermal amplification, helicase-dependent amplification, strand displacement amplification). Additional suitable methods for determining expression levels of nucleic acids include CRISPR-based detection methods, sequencing methods (e.g. Sanger sequencing, RNA sequencing, pyrosequencing, ion torrent sequencing, sequencing-by-synthesis, droplet-digital sequencing, sequencing-by-synthesis, etc.), Northern blotting and Southern blotting. Suitable sequencing technologies are reviewed in, for example, Zhong et al., Ann Lab Med. 2021 Jan; 41(1): 25-43, and Slatko et al., Curr Protoc Mol Biol. 2018 Apr; 122(1): e59., the entire contents of each of which are incorporated herein by reference.

Techniques for measuring gene expression include, for example, gene expression assays with or without the use of gene chips, which are described in Onken et al., J Molec Diag 12(4): 461-468 (2010); and Kirby et al., Adv Clin Chem 44: 247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, Mass.).

Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLOS ONE 3(7): e2535. Additional exemplary methods for determining expression levels of protein include proteomic analysis, or the systematic identification and quantification of proteins of a particular biological system. Mass spectrometry, protein chips, and protein microarrays may be used for protcomic analysis.

In some embodiments, provided herein is a method comprising obtaining a sample from a microenvironment of a synthetic scaffold implanted in a subject, and measuring an expression level of a panel of genes in the sample. In some embodiments, the panel of genes comprises two or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises six or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises 10 or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises less than 100 genes. In some embodiments, the panel of genes comprises less than 100, less than 90, less than 80, less than 70, less than 60, less than 50, less than 40, less than 30, or less than 20 genes.

In some embodiments, the subject is diagnosed with or at risk of having cancer. In some embodiments, the subject is a candidate for therapy with an immune checkpoint inhibitor. In some embodiments, the subject has not received treatment with an immune checkpoint inhibitor. In some embodiments, the method further comprises providing an immune checkpoint inhibitor to the subject when the expression level of one or more of Pmepa11, Gm50439, Tnfsf9, and Gar1is decreased relative a control level or a cutoff level. For example, in some embodiments the method comprises providing an immune checkpoint inhibitor to the subject when the expression level of two or more of Pmepa11, Gm50439, Tnfsf9, and Gar1is decreased relative a control level or a cutoff level. As another example, in some embodiments the method comprises providing an immune checkpoint inhibitor to the subject when the expression level of three or more of Pmepa11, Gm50439, Tnfsf9, and Gar1is decreased relative a control level or a cutoff level. In some embodiments, the method comprises providing an immune checkpoint inhibitor to the subject when the expression level of Pmepa11, Gm50439, Tnfsf9, and Gar1is decreased relative a control level or a cutoff level.

In some embodiments, the method comprises providing an immune checkpoint inhibitor to the subject when the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level. In some embodiments, the method comprises providing an immune checkpoint inhibitor to the subject when the expression level of two or more of, three or more of, four or more of, five or more of, six or more of, seven or more of, eight or more of, nine or more of, 10 or more of, or 11 or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level. In some embodiments, the method comprises providing an immune checkpoint inhibitor to the subject when the expression level of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level.

In some embodiments, the method comprises providing an alternative cancer treatment to the subject when the expression level of one or more of Pmepa11, Gm50439, Tnfsf9, and Gar1is increased relative a control level or a cutoff level. In some embodiments, the method comprises providing an alternative cancer treatment to the subject when the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level. In some embodiments, the method comprises providing an alternative cancer treatment to the subject when the expression level of one or more of Pmepa11, Gm50439, Tnfsf9, and Gar1is increased relative a control level or a cutoff level and the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level. In some embodiments, provided herein are methods of predicting response to an immune checkpoint inhibitor in a subject. In some embodiments, the subject is diagnosed with or at risk of having cancer and has not received treatment with an immune checkpoint inhibitor. In some embodiments, the method comprises obtaining a sample from a microenvironment of a synthetic scaffold implanted in a subject, measuring an expression level or amount of at least one RNA, at least one gene, at least one cell type, and/or at least one protein in the sample, and predicting response to the immune checkpoint inhibitor based upon the measured expression level or amount of the at least one RNA, at least one gene, at least one cell type, and/or at least one protein in the sample. In some embodiments, the method further comprises comprising comparing the expression level or amount of the at least one RNA, at least one gene, at least one cell type, and/or at least one protein in the sample to a control level or a cutoff level. In some embodiments, the method comprises measuring the expression level of a panel of genes as described herein. In some embodiments, the method comprises measuring the expression level of one or more genes (e.g. one or more, two or more, three or more, four or more, five or more, six or more, seven or more, eight or more, nine or more, 10 or more, 11 or more, 12 or more, 13 or more, 14 or more, 15 or more, or 16 or more genes) selected from Pmepa 1, Gm50439, Tnfsf 9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the method comprises determining whether the subject is likely to be sensitive or resistant to treatment with an immune checkpoint inhibitor based upon the expression levels of the one or more genes measured in the sample.

In some embodiments, the method comprises determining that the subject is likely to be sensitive to treatment with an immune checkpoint inhibitor when the expression level of one or more of Pmepa11, Gm50439, Tnfsf9, and Gar1is decreased relative a control level or a cutoff level. In some embodiments the method comprises determining that the subject is likely to be sensitive to treatment with an immune checkpoint inhibitor when the expression level of two or more of Pmepa11, Gm50439, Tnfsf9, and Gar1is decreased relative a control level or a cutoff level. In some embodiments the method comprises determining that the subject is likely to be sensitive to treatment with an immune checkpoint inhibitor when the expression level of three or more of Pmepa11, Gm50439, Tnfsf9, and Gar1is decreased relative a control level or a cutoff level. In some embodiments the method comprises determining that the subject is likely to be sensitive to treatment with an immune checkpoint inhibitor when the expression level of Pmepa11, Gm50439, Tnfsf9, and Gar1is decreased relative a control level or a cutoff level.

In some embodiments, the method comprises determining that the subject is likely to be sensitive to treatment with an immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level.

In some embodiments, the method comprises determining that the subject is likely to be sensitive to treatment with an immune checkpoint inhibitor when the expression level of one or more of, two or more of, three or more of, four or more of, five or more of, six or more of, seven or more of, eight or more of, nine or more of, 10 or more of, 11 or more of, 12 or more of, 13 or more of, 14 or more of, 15 or more of, or 16 or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level.

In some embodiments, the method comprises determining that the subject is likely to be resistant to treatment with an immune checkpoint inhibitor when the expression level of one or more of Pmepa11, Gm50439, Tnfsf9, and Gar1is increased relative a control level or a cutoff level. For example, in some embodiments the method comprises determining that the subject is likely to be resistant to treatment with an immune checkpoint inhibitor when the expression level of two or more of Pmepa11, Gm50439, Tnfsf9, and Gar1is increased relative a control level or a cutoff level. In some embodiments the method comprises determining that the subject is likely to be resistant to treatment with an immune checkpoint inhibitor when the expression level of three or more of Pmepa11, Gm50439, Tnfsf9, and Gar1is increased relative a control level or a cutoff level. In some embodiments the method comprises determining that the subject is likely to be resistant to treatment with an immune checkpoint inhibitor when the expression level of Pmepa11, Gm50439, Tnfsf9, and Gar1is increased relative a control level or a cutoff level.

In some embodiments, the method comprises determining that the subject is likely to be resistant to treatment with an immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level. In some embodiments, the method comprises determining that the subject is likely to be resistant to treatment with an immune checkpoint inhibitor when the expression level of two or more of, three or more of, four or more of, five or more of, six or more of, seven or more of, eight or more of, nine or more of, 10 or more of, 11 or more of, 12 or more of, 13 or more of, 14 or more of, 15 or more of, or 16 or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level.

In some embodiments, the method further comprises providing an immune checkpoint inhibitor to the subject determined as likely to be sensitive to treatment with an immune checkpoint inhibitor, or providing an alternative cancer treatment to the subject identified as being likely to be resistant to treatment with the immune checkpoint inhibitor.

Any of the methods described herein may comprise analyzing cells obtained from the sample. For example, cell type analysis may be performed on the sample obtained from the subject, and compared to cell types present in a control sample (e.g. a sample obtained from the subject prior to treatment with the immune checkpoint inhibitor, or a sample obtained from a control subject not afflicted with cancer). In some embodiments, the cell types present in the sample obtained following treatment with an immune checkpoint inhibitor may be assessed to determine whether the subject is sensitive to the immune checkpoint inhibitor (e.g. exhibits a positive response to the immune checkpoint inhibitor). In some embodiments, the cell types present in the sample may be assessed in a subject that has not received treatment with an immune checkpoint inhibitor and used to predict likelihood of the subject being sensitive to treatment with an immune checkpoint inhibitor.

Various methods for analyzing cells in the sample may be used. In some embodiments, methods for analyzing cells may comprise separating cells into cell type specific subpopulations. In some embodiments, the sample containing the cells of interest may be contacted with one or more labels (e.g. antibodies, each antibody comprising a fluorescent molecule) and the label can be used to sort and/or identify cell types of interest. For example, cells may be separated into cell types by known techniques, including, for example, flow cytometry, fluorescent-assisted cell sorting (FACS), magnetic-assisted cell sorting, histological techniques, e.g., fluorescent immunohistochemistry, or multiplexed fluorescent imaging technologies. In exemplary aspects, the methods comprise monitoring the cell populations over time. In exemplary aspects, the methods comprise measuring or quantifying the different cell populations in the sample, in addition to or instead of measuring a level of expression of a gene, an RNA or a protein, in the sample.

In some embodiments, the methods comprise performing cell type analysis to determine an amount of at least one cell type in the sample. In some embodiments, the methods comprise performing cell type analysis to determine an amount of at least two cell types in the sample. In some embodiments, the methods comprise calculating a ratio of a first cell type: a second cell type in the sample. In some embodiments, the amount or ratio is used to predict sensitivity to treatment with an immune checkpoint inhibitor. In some embodiments, the amount or ratio is used to determine sensitivity to the immune checkpoint inhibitor in a subject undergoing treatment with an immune checkpoint inhibitor.

In some embodiments, the cell types assessed include one or more of B-cells, NK-cells, neutrophils, T-cells, and macrophages. In some embodiments, the methods comprise measuring an amount of one or more cell types selected from B-cells, NK-cells, neutrophils, T-cells, and macrophages in the sample. In some embodiments, an increased amount of one or more cell types relative to a control indicates that the subject is resistant to treatment with an immune checkpoint inhibitor. For example, in some embodiments an increased amount of B-cells and/or NK-cells indicates that the subject is resistant to treatment with an immune checkpoint inhibitor. In some embodiments, the methods comprise calculating a ratio of the amount of neutrophils: T-cells, neutrophils: B-cells, neutrophils: NK-cells, and/or macrophages: NK-cells in the sample. In some embodiments, an increased ratio of neutrophils: T-cells, neutrophils: B-cells, neutrophils: NK-cells, and/or macrophages: NK-cells indicates that the subject is resistant to or likely to be resistant to treatment with an immune checkpoint inhibitor. In some embodiments, an increased ratio of neutrophils: T-cells, neutrophils: B-cells, neutrophils: NK-cells, and/or macrophages: NK-cells indicates that the subject receiving treatment with an immune checkpoint inhibitor is resistant to the treatment with an immune checkpoint inhibitor.

In some embodiments an increased ratio of neutrophils: T-cells indicates that the subject receiving treatment with an immune checkpoint inhibitor is resistant to the treatment with an immune checkpoint inhibitor. In some embodiments an increased ratio of neutrophils: B-cells indicates that the subject receiving treatment with an immune checkpoint inhibitor is resistant to the treatment with an immune checkpoint inhibitor. In some embodiments an increased ratio of neutrophils: NK-cells indicates that the subject receiving treatment with an immune checkpoint inhibitor is resistant to the treatment with an immune checkpoint inhibitor. In some embodiments an increased ratio of macrophages: NK-cells indicates that the subject receiving treatment with an immune checkpoint inhibitor is resistant to the treatment with an immune checkpoint inhibitor.

In some embodiments, provided herein is a method comprising obtaining a sample from a microenvironment of a synthetic scaffold implanted in a subject; and measuring an expression level of a panel of genes in the sample and/or measuring an amount of one or more cell types in the sample. In some embodiments, the panel of genes comprises two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1. In some embodiments, the panel of genes comprises two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the panel of genes comprises two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, the panel of genes comprises six or more genes. In some embodiments, the panel of genes comprises 10 or more genes. In some embodiments, the panel of genes comprises 16 or more genes. In some embodiments, the panel of genes comprises less than 100 genes. In some embodiments, the panel of genes comprises less than 100, less than 90, less than 80, less than 70, less than 60, less than 50, less than 40, less than 30, or less than 20 genes. In some embodiments, the more cell types selected from B-cells, NK-cells, neutrophils, T-cells, and macrophages in the sample.

In some embodiments, subject is diagnosed with or at risk of having cancer and has received treatment with an immune checkpoint inhibitor. In some embodiments, the method further comprises providing an alternative cancer treatment to the subject when the amount of B-cells is increased relative to a control level or a cutoff level. In some embodiments, the method further comprises providing an alternative cancer treatment to the subject when the amount of NK-cells is increased relative to a control level or a cutoff level. In some embodiments, the method further comprises providing an alternative cancer treatment to the subject when a ratio of the amount of neutrophils: T-cells, neutrophils: B-cells, neutrophils: NK-cells, and/or macrophages: NK-cells is decreased relative to a control.

In some embodiments, provided herein are methods of monitoring response to treatment with an immune checkpoint inhibitor in a subject. In some embodiments, the subject is diagnosed with or at risk of having cancer and has received treatment with an immune checkpoint inhibitor. In some embodiments, the method comprises obtaining a sample from a microenvironment of a synthetic scaffold implanted in a subject; and measuring an expression level or amount of at least one RNA, at least one gene, at least one cell type, and/or at least one protein in the sample. In some embodiments, the method further comprises comparing the expression level or amount of the at least one RNA, at least one gene, at least one cell type, and/or at least one protein in the sample to a control level or a cutoff level. In some embodiments, the method further comprises determining whether the subject is sensitive to (e.g. exhibits a positive response to) or resistant to (e.g. does not exhibit a positive response to) treatment with the immune checkpoint inhibitor based upon the expression level or amount of the at least one RNA, at least one gene, at least one cell type, and/or at least one protein in the sample.

In some embodiments, the method of monitoring response to treatment with an immune checkpoint inhibitor comprises measuring the expression level of a panel of genes as described herein. In some embodiments, the method comprises measuring the expression level of one or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1, measuring the expression level of two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3, measuring the expression level of two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2; and/or measuring the amount of one or more cell types selected from B-cells, NK-cells, neutrophils, T-cells, and macrophages in the sample. In some embodiments, the method comprises measuring the expression level of one or more genes, two or more genes, three or more genes, four or more genes, five or more genes, six or more genes, seven or more genes, eight or more genes, nine or more genes, 10 or more genes, 11 or more genes, 12 or more genes, 13 or more genes, 14 or more genes, 15 or more genes, or 16 or more genes. In some embodiments, the method comprises measuring the amount of one or more cell types selected from B-cells, NK-cells, neutrophils, T-cells, and macrophages in the sample. In some embodiments, the method comprises measuring the expression level of one or more genes and measuring the amount of one or more cell types selected from B-cells, NK-cells, neutrophils, T-cells, and macrophages in the sample.

In some embodiments, the method comprises determining whether the subject is sensitive or resistant to treatment with the immune checkpoint inhibitor based upon the expression level or amount of the at least one RNA, at least one gene, at least one cell type, and/or at least one protein in the sample.

In some embodiments, the method comprises determining that the subject is sensitive to the treatment with the immune checkpoint inhibitor when the expression level of one or more of Hs6st2, Gm48732, Gm50393, Gbp2, Rtp4, and Gbp3 is increased relative a control level or a cutoff level. In some embodiments, the method comprises determining that the subject is resistant to the treatment with the immune checkpoint inhibitor when the expression level of one or more of Hs6st2, Gm48732, Gm50393, Gbp2, Rtp4, and Gbp3 is decreased relative a control level or a cutoff level. In some embodiments, the method comprises determining that the subject is sensitive to the treatment with the immune checkpoint inhibitor when the expression level of one or more of Tpsab1, Surf1. Rnf144a, Stfa2, Crlf1, Sprtn, Sh3g13, Nupl1, Tlr12, and Alms1 is decreased relative to a control level or a cutoff level. In some embodiments, the method comprises determining that the subject is resistant to the treatment with the immune checkpoint inhibitor when the expression level of one or more of Tpsab1, Surf1. Rnf144a, Stfa2, Crlf1, Sprtn, Sh3g13, Nupl1, Tlr12, and Alms1 is increased relative to a control level or a cutoff level.

In some embodiments, the method comprises determining that the subject is sensitive to the treatment with the immune checkpoint inhibitor when the expression level of one or more of Slc7a14, Dkk2, Fdps, Trib1, Pagr1a, Csf1, Ptgs2, Gm50393, H2.M2, Gm19195, Sycp2, X2810457G06Rik, and Gm48996 is increased relative to a control level or a cutoff level. In some embodiments, the method comprises determining that the subject is resistant to the treatment with the immune checkpoint inhibitor when the expression level of one or more of Slc7a14, Dkk2, Fdps, Trib1, Pagr1a, Csf1, Ptgs2, Gm50393, H2.M2, Gm19195, Sycp2, X2810457G06Rik, and Gm48996 is decreased relative to a control level or a cutoff level. In some embodiments, the method comprises determining that the subject is sensitive to the treatment with the immune checkpoint inhibitor when the expression level of one or more of Slc4a4, Dtd2, Lvrn, Rnf144a, Zic4, Nupl1, Cd177, Stfa2, and Ripply3 is decreased relative to a control level or a cutoff level. In some embodiments, the method comprises determining that the subject is resistant to the treatment with the immune checkpoint inhibitor when the expression level of one or more of Slc4a4, Dtd2, Lvrn, Rnf144a, Zic4, Nupl1, Cd177, Stfa2, and Ripply3 is increased relative to a control level or a cutoff level. In some embodiments, the method comprises determining that the subject is sensitive to treatment with the immune checkpoint inhibitor when the expression level of one or more of Gm45290, Timm44, Gm19056, Pla2g12a, Gm9118, Lhx6, Tex22, 8430429K09Rik, Gm6361, 530010L16Rik, Gm18362, and Ptgs2 is increased relative to a control level or a cutoff level. In some embodiments, the method comprises determining that the subject is resistant to the treatment with the immune checkpoint inhibitor when the expression level of one or more of Gm45290, Timm44, Gm19056, Pla2g12a, Gm9118, Lhx6, Tex22, 8430429K09Rik, Gm6361, 530010L16Rik, Gm18362, and Ptgs2 is decreased relative to a control level or a cutoff level. In some embodiments, the method comprises determining that the subject is sensitive to the treatment with the immune checkpoint inhibitor when the expression level of one or more of Chsy1, Sprint, Gm18935, Rpl18.ps2, Tlr12, 2900078111Rik, Crlf1, Wdr66, and Syde22 is decreased relative to a control level or a cutoff level. In some embodiments, the method comprises determining that the subject is resistant to the treatment with the immune checkpoint inhibitor when the expression level of one or more of Chsy1, Sprint, Gm18935, Rpl18.ps2, Tlr12, 2900078111Rik, Crlf1, Wdr66, and Syde22 is increased relative to a control level or a cutoff level. The signature gene Grip1 is down-regulated in resistant mice, but changes from lowly up-regulated to moderately down-regulated to finally strongly up-regulated in sensitive mice over the course of treatment.

In some embodiments, the method comprises determining that the subject is resistant to the treatment with the immune checkpoint inhibitor when the amount of B-cells is increased relative to a control level or a cutoff level. In some embodiments, the method comprises determining that the subject is resistant to the treatment with the immune checkpoint inhibitor when the amount of NK-cells is increased relative to a control level or a cutoff level. In some embodiments, the method comprises determining that the subject is resistant to the treatment with the immune checkpoint inhibitor when a ratio of the amount of neutrophils: T-cells, neutrophils: B-cells, neutrophils: NK-cells, and/or macrophages: NK-cells is decreased relative to a control. In some embodiments, the method comprises providing an alternative cancer treatment to the subject identified as resistant to treatment with the immune checkpoint inhibitor. In some embodiments, the methods described herein comprise providing treatment to a subject. In some embodiments, the methods comprise providing to a subject an immune checkpoint inhibitor. For example, in some embodiments the methods comprise providing an immune checkpoint inhibitor to a subject identified as sensitive to or likely sensitive to an immune checkpoint inhibitor. In some embodiments the methods comprise providing an immune checkpoint inhibitor to a subject identified as having increased or decreased expression of given genes in a panel. In some embodiments the methods comprise providing an immune checkpoint inhibitor to a subject identified as having increased or decreased amount of a given cell type, or identified as having a certain ratio of two cell types. The immune checkpoint inhibitor may be any suitable agent that target checkpoint proteins including PD-1, PD-L1, B7-1/B7-2, LAG-3, and CTLA-4. For example, the immune checkpoint inhibitor may be an antibody that binds PD-1, PD-L1, B7-1/B7-2, LAG-3, or CTLA-4. For example, the immune checkpoint inhibitor may be an antibody that binds PD-1, PD-L1, B7-1/B7-2, LAG-3, or CTLA-4. Exemplary immune checkpoint inhibitors include, for example PD-1 inhibitors (e.g. pembrolizumab, nivolumab, cemiplimab), PD-L1 inhibitors (e.g. atezolizumab, avelumab, durvalumab), CTLA-4 inhibitors (ipilimumab), LAG-3 inhibitors (relatlimab), or combinations thereof.

In some embodiments, the methods comprise providing an alternative cancer treatment to the subject. For example, in some embodiments the methods comprise providing an alternative cancer treatment to a subject identified as resistant or likely resistant to an immune checkpoint inhibitor. In some embodiments the methods comprise providing an alternative cancer treatment to a subject identified as having increased or decreased expression of given genes in a panel. In some embodiments the methods comprise providing an alternative cancer treatment to a subject identified as having increased or decreased amount of a given cell type, or identified as having a certain ratio of two cell types. The alternative cancer treatment may comprise any suitable alternative therapy other than immune checkpoint inhibitors, including, for example, surgery, chemotherapy, radiation therapy, bone marrow transplant, immunotherapy, hormone therapy, targeted drug therapy, cyroablation, and combinations thereof.

In some aspects, provided herein are panels of genes (i.e. gene panels). In some embodiments, the panel of genes comprises at least 2, at least 3, at least 4, at least 5, at least 6, at least 7, at least 8, at least 9, at least 10, at least 11, at least 12, at least 13, at least 14, at least 15, or at least 16 genes. In some embodiments, the panel of genes comprises less than 100 genes. In some embodiments, the panel of genes comprises between 2 and 100 genes. For example, in some embodiments the panel of genes comprises at least 10 but less than 100 genes. In some embodiments, the panel of genes comprises two or more of Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises three or more of, four or more of, five or more of, six or more of, seven or more of, eight or more of, nine or more of, 10 or more of, 11 or more of, 12 or more of, 13 or more of, 14 or more of, 15 or more of, or each of Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the panel of genes comprises two or more of Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1. In some embodiments, the panel of genes comprises three or more of, four or more of, five or more of, six or more of, seven or more of, eight or more of, nine or more of, 10 or more of, 11 or more of, 12 or more of, 13 or more of, 14 or more of, 15 or more of, or each of Hs6st2, Gm48732, Gm50393, Gbp 2, Rtp4, Gbp3, Tpsab1, Surf1, Rnf144a, Stfa2, Crlf1, Sprtn, Sh3g13, Nupl1, Tlr12, and Alms1. In some embodiments, the panel of genes comprises two or more of Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the panel of genes comprises three or more of, four or more of, five or more of, six or more of, seven or more of, eight or more of, nine or more of, 10 or more of, 11 or more of, 12 or more of, 13 or more of, 14 or more of, 15 or more of, or each of Slc7a14, Dkk 2, Fdps, Trib 1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the panel of two or more genes comprises two or more of Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, the panel of genes comprises three or more of, four or more of, five or more of, six or more of, seven or more of, eight or more of, nine or more of, 10 or more of, 11 or more of, 12 or more of, 13 or more of, 14 or more of, 15 or more of, or each of Chsy 1, Sprtn, Gm18935, Timm44, Gm19056, Rp118.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2.

In some aspects, provided herein are kits. In some embodiments, provided herein is a kit comprising a synthetic scaffold described herein. The kit may comprise additional components, such as additional materials required for implanting the scaffold in the subject, collecting the scaffold or a portion thereof from the subject (e.g. tweezers, microneedles, tubes, etc.), and analyzing expression of one or more genes, nucleic acids, or proteins in the sample. For example, the kit may additionally comprise components necessary for analysis of gene expression or analysis of RNA expression and/or analysis of protein expression in the sample (e.g. primers, probes, antibodies, buffers, inhibitors, stabilizers, salts, denaturants, and other suitable reagents). In some embodiments, the kit comprises instructions for use. In some embodiments, the kits comprise reagents for detection of a panel of genes as described herein. In some embodiments, provided herein is a kit comprising reagents for detection of a panel of genes in a sample, the panel of genes comprising two or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12. In some embodiments, the kit comprises reagents for detection of a panel of genes comprising less than 100 genes (e.g. less than 100, less than 90, less than 80, less than 70, less than 60, less than 50, less than 40, less than 30, or less than 20 genes). In some embodiments, the kit finds use in a method of predicting response to an immune checkpoint inhibitor in a subject as described herein.

In some embodiments, the kit comprises reagents for detection of a panel of genes comprising two or more genes selected from two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms. In some embodiments, the kit comprises reagents for detection of a panel of genes comprising two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3. In some embodiments, the kit comprises reagents for detection of a panel of genes comprising two or more genes selected from Chsy1, Sprtn, Gm18935, Timm44, Gm19056, Rp118.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2. In some embodiments, the panel of genes comprises six or more genes. In some embodiments, the panel of genes comprises 10 or more genes. In some embodiments, the panel of genes comprises 16 or more genes.

In some embodiments, the kit comprises reagents for detection of a panel of genes comprising less than 100 genes (e.g. less than 100, less than 90, less than 80, less than 70, less than 60, less than 50, less than 40, less than 30, or less than 20 genes). In some embodiments, the kit finds use in a method of monitoring response to treatment with an immune checkpoint inhibitor in a subject as described herein. In some embodiments, monitoring response to the treatment with the immune checkpoint inhibitor in the subject comprises contacting a sample obtained from a microenvironment of a synthetic scaffold implanted in the subject with the reagents for detection of the panel of genes. In some embodiments, the sample is obtained about 1-28 days after receiving the treatment with the immune checkpoint inhibitor. In some embodiments, the sample is obtained about 7-21 days after receiving the treatment with the immune checkpoint inhibitor.

### EXAMPLE 1

### Methods

### Microporous PCL Scaffold Fabrication and Subcutaneous Implantation

To fabricate microporous implants, polycaprolactone (PCL) was mixed with a salt porogen (NaCl, 250-425 μm), pressed into molds (5mm wide, 2 mm thick), the polymer sintered at 135 °C, and porogen-leached as previously described (International Immunopharmacology. 58 (2018) 125-135; Nat Med. 25(2019 ) 920-928)). PCL implants were disinfected and stored at −80 °C until surgery. Immunologic niches were implanted in the dorsal subcutaneous space of 8-week old female Balb/c mice (Jackson Laboratories). All procedures were performed in accordance with the institutional guidelines and protocols approved by the University of Michigan Institutional Animal Care and Use Committee. Mice were anesthetized with isofluorane prior to subcutaneous implantation with PCL implants. Mice received subcutaneous injections of carprofen (5 mg/kg) immediately before surgery and 24 hours after surgery.

### Tumor Cell Culture and Orthotropic Inoculations

Orthotopic inoculation of tumor cells was performed 2 weeks after immunologic niche implantation. 4T1-luc2-tdTomato murine triple negative breast cancer cells (PerkinElmer) were cultured in RPMI 1640 Medium (Thermo Fisher Scientific) containing 10% fetal bovine serum (FBS, INFO) for 5 days (37 °C, 5% CO2, #% O2) prior to inoculation. Tumor cells were enzymatically lifted from the tissue culture flask with trypsin (INFO probably Sigma) for 10 minutes at 37 °C and resuspended in culture medium. Cells were centrifuged at 500× g for 5 minutes and resuspended in Dulbecco's phosphate buffered saline (DPBS) at a concentration of 40E6 cells/mL. The tumor cell line was previously confirmed to be pathogen free and authenticated by short tandem repeat DNA analysis and compared to the ATCC STR profile database (DDC Medical). Orthotopic inoculations were performed by injecting 50 μL of the cell suspension, containing 2E6 4T1 tumor cells, to the fourth right mammary fat pad of 12-week-old female Balb/c mice (Jackson Laboratory, 000651).

### Anti-PD-1 Administration

In VivoMab anti-mouse anti-PD-1 (CD279) antibody (BE0146, clone RMP1-14, BioXCell) and isotype control (BE0089, In VivoMAb rat IgG2a isotype control, BioXCell) were diluted in DPBS to a final concentration of 1 mg/mL immediately prior to intraperitoneal (IP) injections. A volume of 100 uL of diluted aPD-1 and isotype control were administered (10 mg/kg) IP on days 9, 11, 13, and 15 post tumor-inoculation.

### Tumor Volume Measurements and Survival Monitoring

Tumor size was recorded using standard electronic calipers (VWR) while mice were anesthetized with 2% v/v% isoflurane. Primary tumor volume was calculated (V=0.5×L×W2, L: length of longest dimension of the tumor, W: length perpendicular to the longest tumor dimension) as previously described [68]. Mice were monitored for tumor size and body conditioning to determine survival. Mice were euthanized if any of the following criteria were met: tumor size of >2 cm in any dimension, ulceration of more than 50% of the visible tumor area, partial paralysis due to tumor invasion of hind limb muscle, labored breathing, ascites, lethargy, or visible weight loss.

### Tissue Isolations

Niche implants were surgically explanted at days 7, 14, and 21 (D7, D14, D21) post-tumor inoculation to study gene expression changes associated with ICB-response. Mice were anesthetized with isoflurane before an incision was made over the surface of the implant. The niche implant and any adherent encapsulating tissue were pulled through the incision and excised, and the incision closed with sutures. Immunologic niche tissues for RNA analyses were flash frozen in isopentane on dry ice and stored at −80 °C. For the flow cytometry analysis, mice were euthanized at study endpoint (D21) and the primary tumor, spleen, and implant isolated. Tissues were placed into PBS and stored on ice.

### RNA Isolation, Purity, Integrity, and Bulk RNA-seq

Explanted immunologic niche tissues were immediately flash frozen in isopentane. Frozen implants were homogenized in Trizol and RNA subsequently isolated from homogenate using the Direct-zol™ RNA Kit (Zymo Research) with DNase 1 treatment. The isolated RNA was diluted to the desired concentration and submitted to the University of Michigan Advanced Genomics Core for analysis with total RNA (ribo-depletion) library preparation and bulk RNA-seq performed on the Illumina NovaSeq™ S4 at PE150 (45-60 million reads/sample). Raw counts, as prepared from demultiplexed fastq files by the Advanced Genomics Core, were converted to normalized counts using DEseq 2 (Cancer Res. 74(2014 ) 104-118).

### Analysis of Gene Expression Differentially Regulated Pathways

Normalized RNA-seq counts were screened to identify differentially expressed genes of interest associated with response to ICB. T-tests were first performed to compare ICB-sensitive and ICB-resistant for each gene. This initial analysis identified the 100's of genes differentially expressed between sensitive and resistant. These screened genes were then probed with elastic net-based coefficient reduction, favoring group selection (α=0.05), with 2000 iterations of leave-one-out cross validation. Multivariate gene signatures were visualized with principal component analysis for dimensionality reduction. For pathway analysis, mouse gene symbols for RNA-seq counts were converted to human gene symbols using the biomaRt package. All computation was performed using R except for the EN-based analysis, which was performed in MATLAB. Gene Set Enrichment Analysis (GSEA) was performed on DESeq2-normalized counts and the corresponding human gene symbols. Normalized enrichment score (NES) values are visualized for the analysis of ICB-sensitive versus ICB-resistant.

### Flow Cytometry

The primary tumor, spleen, and immunologic niche tissue were mechanically and enzymatically digested. Tissues were processed through a 70 μm cell strainer (Corning) to filter. Single cell suspensions of were then prepared by erythrocyte lysis in ACK buffer (Fisher, #A 1049201) and washed in DPBS (2 mM EDTA, 0.5% bovine serum albumin) by centrifuging at 500× g for 5 minutes. Cells were equally split into two tubes to enable staining and analysis of innate immune cells and lymphocytes from the same tissues. Each tube was treated with anti-CD16/32 (Biolegend) to block nonspecific staining. The innate immune cell panel was stained with AF700 anti-CD45, BV510 anti-CD11b, PEcy7 anti-F4/80, PacBlue anti-Ly6G, and FITC anti-Ly6C (Biolegend) antibodies. The lymphocyte panel was stained with AF700 anti-CD45, FITC anti-CD8, V500 anti-CD4, PacBlue anti-CD19, and PECy7 anti-CD49b (Biolegend) antibodies. All samples were stained with DAPI for viability and analyzed on the BioRad flow cytometer Cytoflex Cell Analyzer. Data analysis was performed using FlowJo (BD).

### Magnetic Activated Cell Sorting of Cell Populations

Single cell suspensions were prepared from explanted immunologic niche tissues and labeled with magnetic microparticle-conjugated antibodies against CD11b (Miltenyi Biotec).

Labelled cells were magnetically sorted. The positive fraction, containing enriched CD11b+ myeloid cells, and the negative fraction, representing non-myeloid cells, were washed by centrifugation at 500× g for 5 minutes. Pelleted cell populations were resuspended in Trizol and stored at −80C until RNA isolations performed.

### Statistical Analysis

Two-tailed unpaired t-tests assuming unequal variance were performed for single comparisons between two conditions, namely ICB-sensitive and ICB-resistance. Median survival and survival curves were analyzed using a simple survival analysis (Kaplan-Meier) with log-rank (Mantel-Cox test) for statistical significance. Following the normalization of RNA-seq gene expressions with DESeq2 (citation), T-tests were performed for single gene comparisons (p<0.05). Differentially expressed genes identified from the T-tests were then parsed with elastic net-based feature identification. For the GSEA analyses, pathways with INESI>1 were considered as being differentially regulated. Prism 9 (GraphPad), Excel (Microsoft), and R were used for performing statistical analyses, with p<0.05 considered to be statistically significant.

Error bars on plotted data are calculated as standard error mean (SEM).

### Results

### Divergent Disease Progression and Survival in Response to Treatment With Checkpoint Blockade Therapy

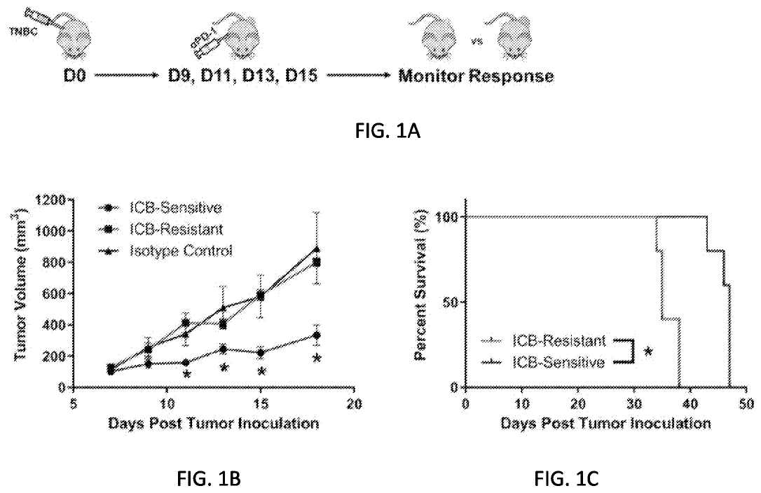

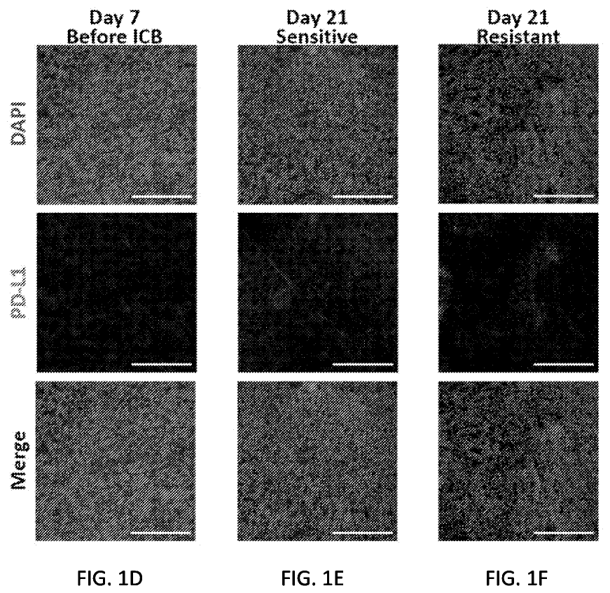

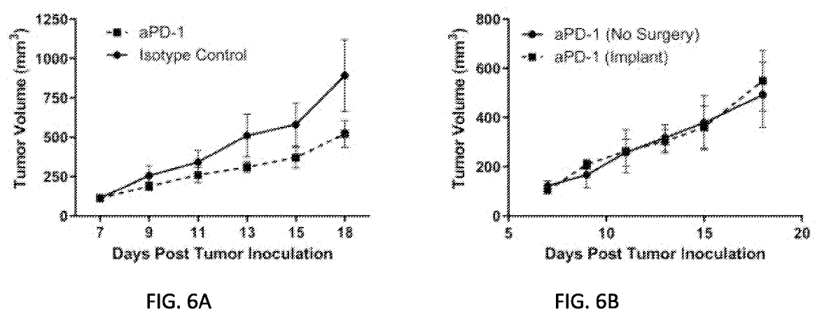

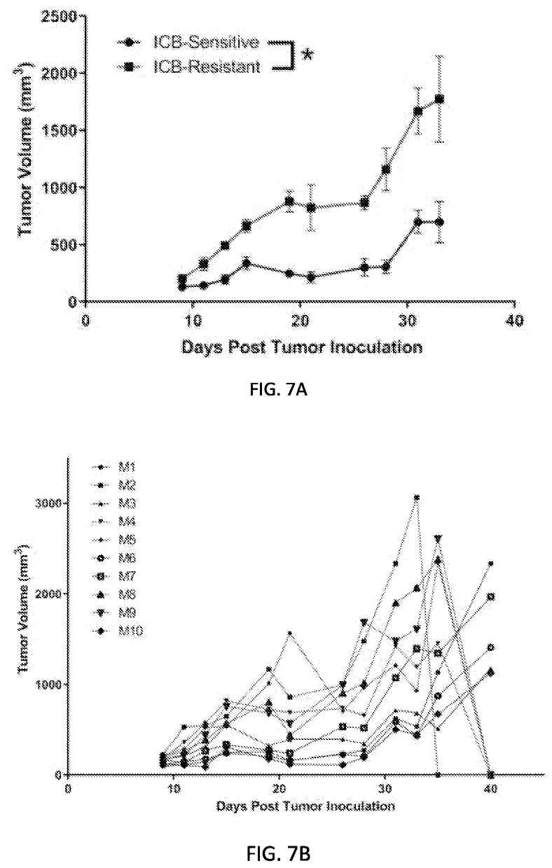

Divergent ICB-responses in a murine model of advanced TNBC were investigated. Balb/c mice were orthotopically inoculated (DO) with triple negative 4T1 tumor cells at the fourth right mammary fat pad. Mice were split into two cohorts and received either intraperitoneal (IP) administrations of aPD-1 or isotype control every other day starting on day 9 post-tumor inoculation, for a total of four doses (D9, D11, D13, D15; FIG. 1A). Waiting for at least a week post-tumor inoculation to initiate ICB allowed for the formation of established primary disease prior to starting treatment. Longitudinal PT volumes were recorded and mice were monitored for survival. While mice receiving aPD-1 trended toward reduced PT growth, no significant difference was found between the aPD-1 and isotype control cohorts (FIG. 6A, p>0.05). The aPD-1 cohort was then stratified based on ICB-response. Mice whose PT growth was less than the established cutoff for fold change in PT volume on D21 post-tumor inoculation, as compared to the baseline (D7), were categorized as ICB-sensitive (FIG. 7). The ICB-resistant mice were categorized as those with a fold change in PT volume above this cutoff. Interestingly, when categorizing the ICB cohort based on ICB-response, ICB-sensitive mice had significantly reduced PT growth versus the ICB-resistant mice on days 11, 13, 15, and 19 post-tumor inoculation (FIG. 1B, p<0.05). Mice that were sensitive to ICB also had significantly reduced PT growth compared to the isotype control cohort, and ICB-resistant mice had indistinguishable progression versus the isotype control (FIG. 1B). In addition to changes in PT growth, ICB-sensitive mice had significantly improved survival compared to the ICB-resistant cohort (FIG. 1C, p<0.05). Prior to treatment, these tumors are PD-L1 positive (FIG. 1D), and PD-L1 expression in the primary tumor cannot distinguish between ICB-sensitivity and resistance (FIG. 1E, F).

Following the observation that tumor-bearing Balb/c mice treated with aPD-1 had divergent PT growth and survival in response to ICB, it was investigated whether these divergent immune responses could be monitored at the immunologic niche by investigating implant-derived gene expressions with bulk RNA sequencing (RNA-seq).

### Gene Expression and Immune Pathways are Differentially Regulated After Therapy Between ICB Sensitivity and Resistance

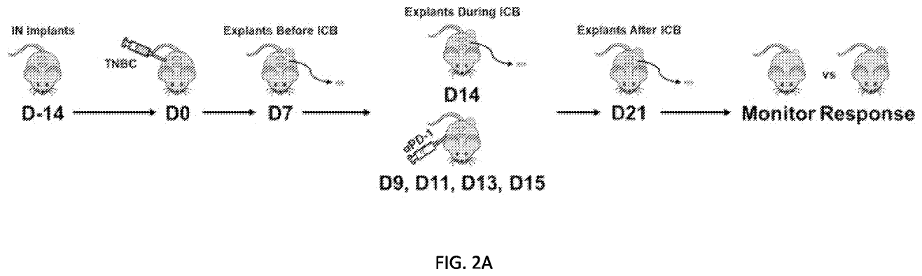

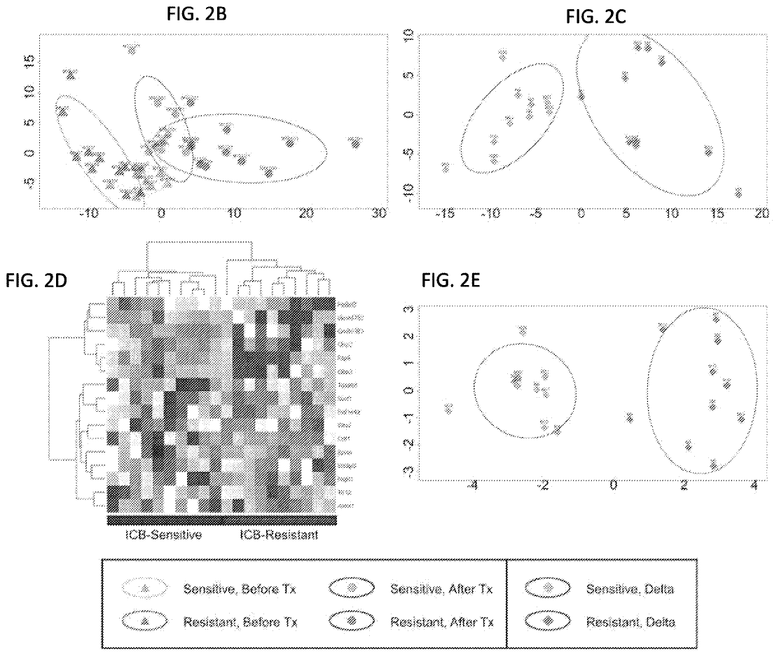

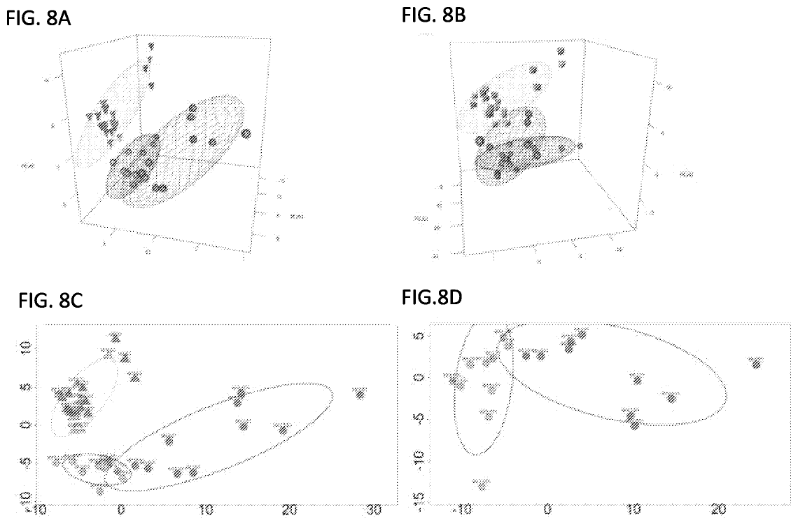

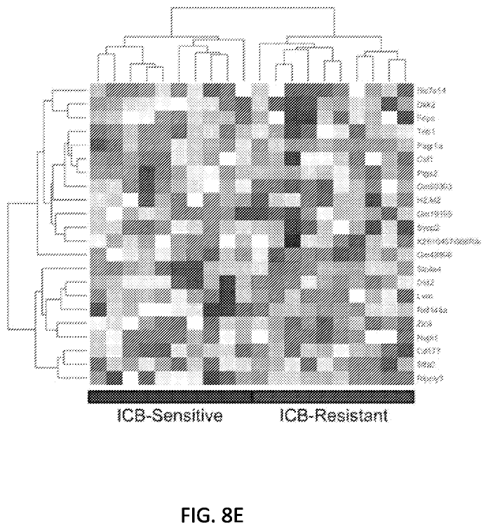

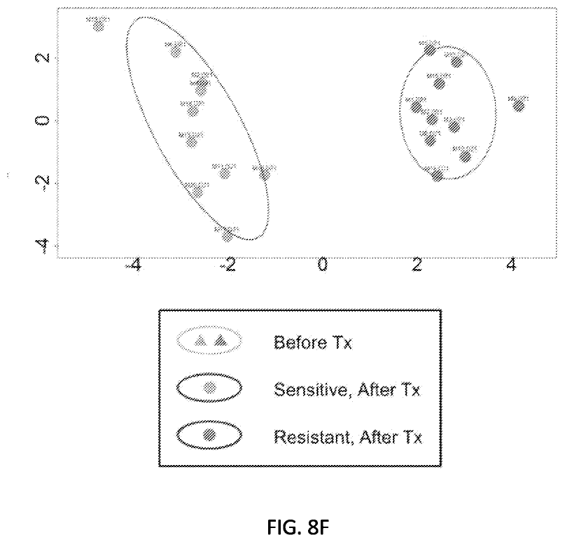

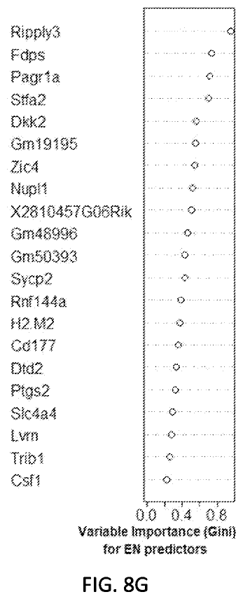

Bulk RNA-seq was performed on RNA from the immunologic niche to investigate implant-derived gene expressions for longitudinally monitoring ICB-response. Microporous polycaprolactone (PCL) scaffold implants (thickness-2 mm, diameter-5 mm) with interconnected pores (250-425 mm) were surgically inserted into the dorsal subcutaneous space of Balb/c mice 14 days prior to tumor inoculation (D-14) to allow for tissue infiltration and integration with the host (FIG. 2A). The microporous architecture facilitates cell colonization throughout the entirety of the implant, and immune changes at the immunologic niche are reflective of dynamics in disease progression. Mice were orthotopically inoculated with 4T1 tumor cells (D0) and an immunologic niche implant was biopsied (D7) once the primary disease was established to analyze the immune response at baseline, prior to initiating ICB therapy (FIG. 2A). Four doses of aPD-1 were administered IP every other day starting on D9 post-tumor inoculation. Niche implants were biopsied at D14 and D21 for analyzing immune changes during therapy (D14) and after the conclusion of ICB therapy (D21) (FIG. 2A). PT volumes were longitudinally recorded and survival monitored to stratify mice into ICB-sensitive and ICB-resistant cohorts. Biopsied immunologic niche tissues were immediately flash frozen in isopentane. Frozen implants were homogenized in Trizol and RNA subsequently isolated from homogenate using the Direct-zol™ RNA Kit. The isolated RNA was diluted to the desired concentration and submitted to the University of Michigan Advanced Genomics Core for analysis with total RNA (ribo-depletion) library preparation and bulk RNA-seq performed on the Illumina NovaSeq™ S4 at PE150 (45-60 million reads/sample). Raw counts, as prepared from demultiplexed fastq files by the Advanced Genomics Core, were converted to normalized counts using DEseq2. To investigate immunologic niche-derived genes for monitoring ICB-response, the bulk RNA-seq data was first screened with T-tests comparing the differential gene expressions after therapy (D21, after Tx). This analysis identified 242 differentially expressed genes (DEGs) between the ICB-sensitive and ICB-resistant cohorts (FIGS. 8A-8C) .

Principal component analysis (PCA) was used for dimensionality reduction to visualize how the cohorts clustered based on the expressions of these 242 DEGs after Tx (FIG. 8A-8C) . As the first two components were found to be responsible for the majority of the variance, only PCA1 and PCA2 were visualized for the remaining analyses (FIGS. 8A-8B) . Elastic net-based coefficient reduction, favoring group selection (α=0.05), was then implemented to identify the most biologically relevant genes from this 242-gene panel. This a value was utilized to retain highly correlated variables, as covarying features are valuable for identifying genes with related mechanistic insight. Cross validation with 2000 random resampling iterations was employed and features that were chosen in more than 85% of iterations were selected to ensure robustness of the identified gene signature. The EN analysis of after Tx DEGs identified a panel of 22 genes (FIG. 8E). Hierarchical clustering of the multivariate gene expressions grouped the cohorts based on their response after therapy (Tx) (FIG. 8E). PCA-based dimensionality reduction of the implant-derived gene expressions from this 22 gene panel clustered mice separately based on ICB-response after Tx (FIG. 8F). Notably, 13/22 of the genes were upregulated in the ICB-sensitive mice, whereas 9/22 of the EN-identified genes were upregulated in the ICB-resistant mice. Ripply3, Fdps, Pagr1a, and Stfa2 account for the most variable importance of the panel in distinguishing ICB response (FIG. 8G). This signature of ICB response after therapy indicates that the IN recapitulates aspects of disease. Collectively, these results indicate that analysis of gene expression at the IN implant immediately after completion of ICB therapy distinguishes sensitivity from resistance at both the pathway and individual gene levels.

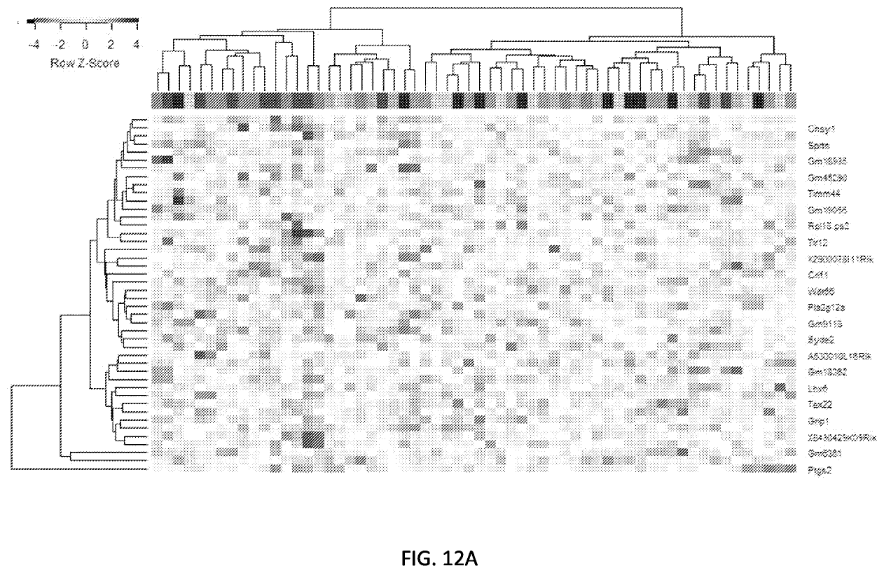

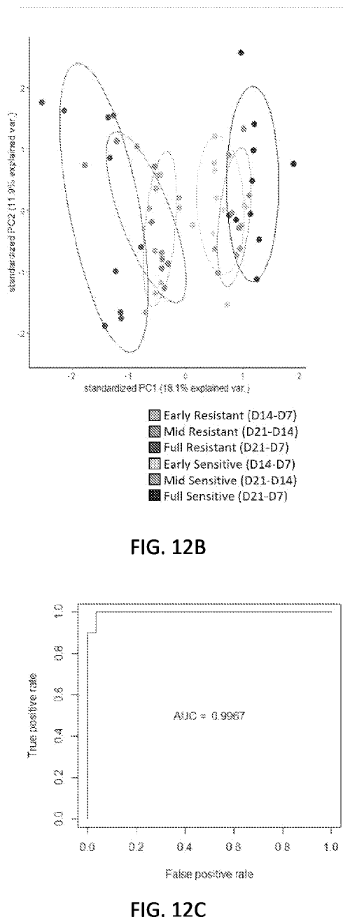

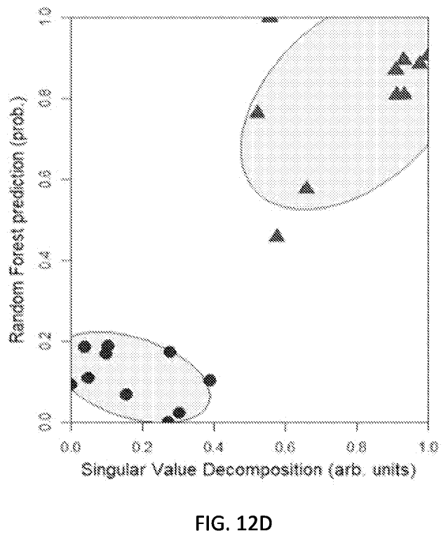

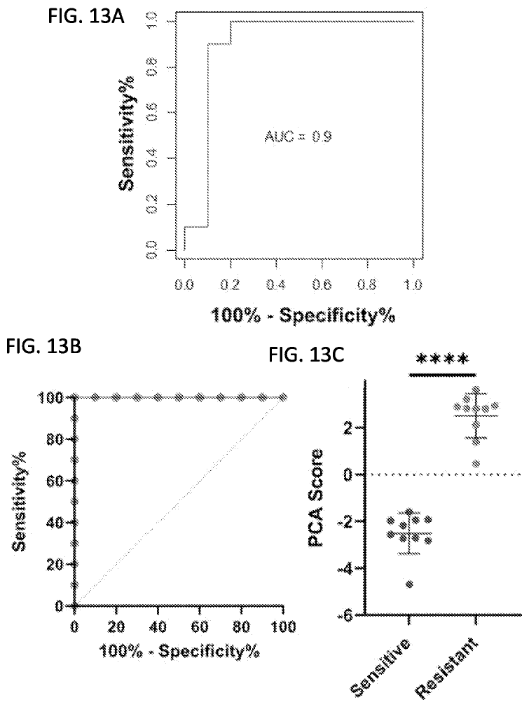

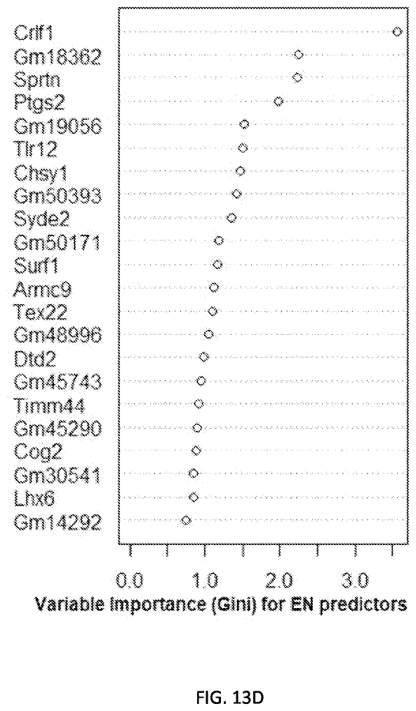

After observing that the gene expressions at the immunologic niche could be monitored after therapy to glean insight into ICB-response, it was investigated whether normalizing the implant-derived gene expressions from each mouse to their 2 baseline before Tx could probe for dynamic changes in ICB-response. Additional analysis was conducted to perform bulk RNAseq to identify differential gene expression at day 7 (before therapy), day 14 (after therapy), and day 21. Results are shown in FIG. 12. A 22-gene signature panel of ICB response across therapy was identified (FIG. 12A). This 22-gene signature panel is referred to as a “serial signature” or a “serial panel”. A more rigorous scoring system involving an unsupervised clustering of samples through singular value decomposition (SVD), and a second supervised method using the bagged-tree Random Forest™ (RF) machine learning algorithm was used. The SVD and RF each assign a score to the gene panel that can be used to assess the ability to distinguish sensitivity and resistance. These scoring metrics separate the two groups relatively well, and the receiver operating characteristic (ROC) curve sensitivity and specificity for identifying ICB response with this serial gene panel indicated a 10% error rate (FIG. 13).

Subsequently, integrating the D21 time point, EN regression and cross validation identified a sparse 22-gene signature panel of ICB response across therapy, and the SVD and RF algorithm exhibited strong separation of the ICB-response cohorts at D21 (FIG. 12D), with the ROC curve being error-free (FIG. 13B, C). Of these 22 genes, 14 had increased expression from baseline (D7) to after treatment among the ICB-sensitive cohort, whereas the remaining 8 genes decreased (FIG. 12A). Differences between conditions were observed between all time points; however, the greatest differences emerged between days 7 and 21. Among the panel, Crlf1 and, to a lesser extent, Gm18362, Sprtn, and Ptgs2 have the greatest variable importance in separating the cohorts (FIG. 13D). Collectively, longitudinal IN-derived gene expression provides the ability to distinguish sensitivity and resistance both during and after ICB treatment.

### Serial Analysis of Gene Expressions Monitors for Unique ICB-response Dynamics

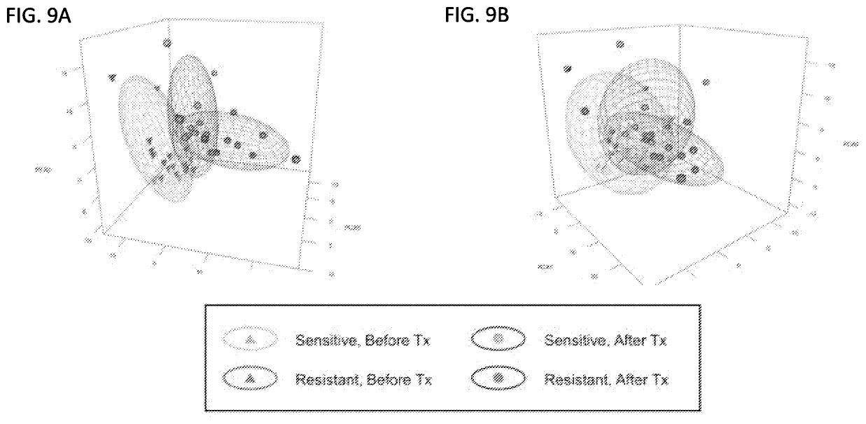

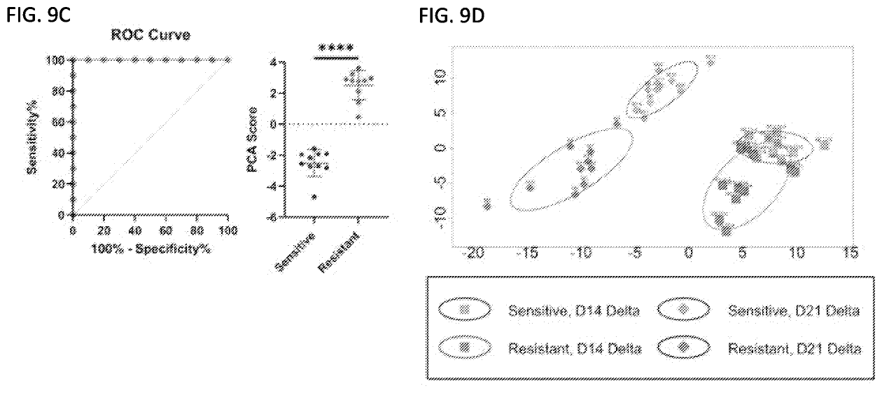

It was tested whether a delta analysis, or normalization to baseline (D7), of the immunologic niche-derived gene expressions could more effectively illuminate dynamics in ICB-response. Delta counts were first calculated by normalizing the DEseq2-normalized counts for each gene after Tx (D21) to the DEseq2-normalized counts before Tx (D7), for each mouse. T-tests were then performed between cohorts for the delta counts of each gene to screen for DEGs (p<0.05). This initial screen identified 237 DEGs between ICB-sensitive and ICB-resistant cohorts (FIGS. 2B-2C). Visualization of the DEseq2-normalized gene expressions by PCA showed separate clustering of the mice not only after Tx, but also before Tx (FIG. 2B). Notably, PCA clustering of the delta counts (expressions normalized to D7) showed separation of the cohorts based on ICB-response after Tx (D21 expressions normalized to D7, FIG. 2C), as well as during Tx (D14 normalized to D7, FIG. 9D). Performance metrics were calculated for sensitivity, specificity, and categorization efficacy for the 237-gene delta-panel (FIG. 9C). The 237-gene delta panel was then analyzed with EN to identify the most relevant DEGs for monitoring ICB-response. Through 2000 iterations of cross validation, the EN analysis of the delta DEGs identified a signature of 16 genes. Excitingly, PCA-based clustering of the 16-gene delta signature showed excellent separation of the ICB-sensitive and ICB-resistant cohorts (FIG. 2E). Hierarchical clustering of the 16-gene panel separated cohorts based on ICB-response (FIG. 2D). Notably, 6/16 genes increased in expression from baseline (D7) to after Tx (D21) among the ICB-sensitive cohort, whereas the remaining 10 genes decreased (FIG. 2D). Performance metrics were calculated for the EN-identified delta-panel (FIG. 9C). The 16-gene panel more effectively separated ICB-sensitive from ICB-resistant than the after panel. It is worth noting that when visualizing the PCA-based clustering of ICB-sensitive and resistant cohorts (using DEseq2-normalized counts versus the delta counts), the delta panel (FIG. 2B) differentially clustered cohorts before Tx, whereas the after panel (FIG. 8C) did not. Slc2a12, Slc5a7, Aldh1a2, and Id3 show the greatest variable importance among this predictive panel. Slc2a12 is upregulated in exosomes derived from patients with gastric cancer and has been correlated with tumor size, stage, lymph node metastasis, and degree of differentiation. Slc5a7 has potential as a biomarkers in the personalized therapy for lung adenocarcinoma and lung squamous cell carcinoma. Aldh1a2 has been associated with tumor suppression and regulation in numerous cancers and has shown prognostic value. Id3 has been noted to govern colon cancer-initiating cell self-renewal through cell-cycle restriction driven by the cell-cycle inhibitor p21 and suppression reduces proliferation rate, invasiveness and anchorage-independent growth. These results demonstrate that analysis of the IN identifies sensitivity or resistance to ICB prior to the initiation of treatment.

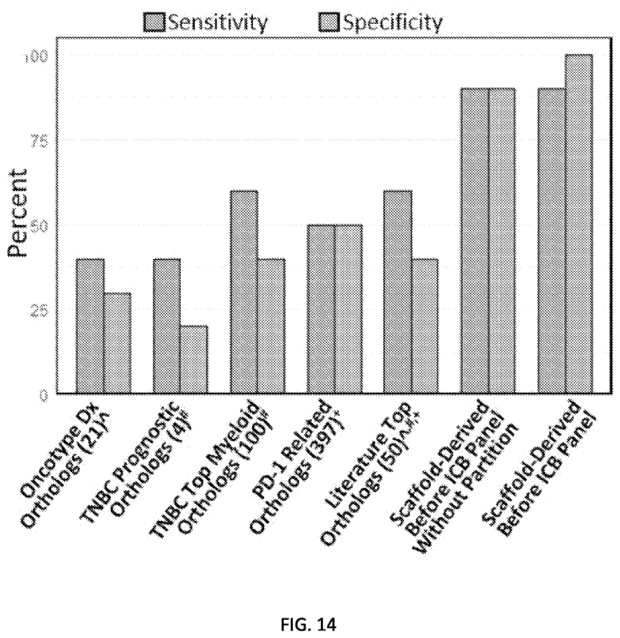

After validating the 16-gene IN signature of before ICB sensitivity, the signature was further compared to potential biomarkers identified in the field (FIG. 14). These comparison transcripts include the murine orthologs of the Oncotype DX® panel, orthologs of transcripts identified as prognostic T cell markers of TNBC immune modulation and myeloid activity (Comput Biol Med. 2023; 161: 107066), and orthologs identified as prognostic of TNBC survival based on PD-1 expression and tumor immune infiltration (Breast Cancer. 2022; 29(4): 666-676). The cross-validation IN signature vastly outperforms each of these tumor-based gene panels in sensitivity and specificity in identifying ICB sensitive and ICB resistant mice. Additionally, the 16-gene panel from the IN derived by partitioning and rigorous cross-validation outperforms a distinct 19-gene panel of pre-treatment ICB sensitivity at the IN without partitioning samples for independent validation. These sensitivity and specificity results collectively indicate that we have derived a gene signature of ICB sensitivity which provides unique information from biomarkers derived at the primary tumor.

Following the exciting findings that implant-derived gene expressions provide dynamic information for monitoring divergent ICB-responses, how inflammatory pathways were being differentially regulated in response to ICB were next investigated.

### Gene Expression and Immune Cell Pathways Are Differentially Regulated Between ICB-Sensitivity and Resistance

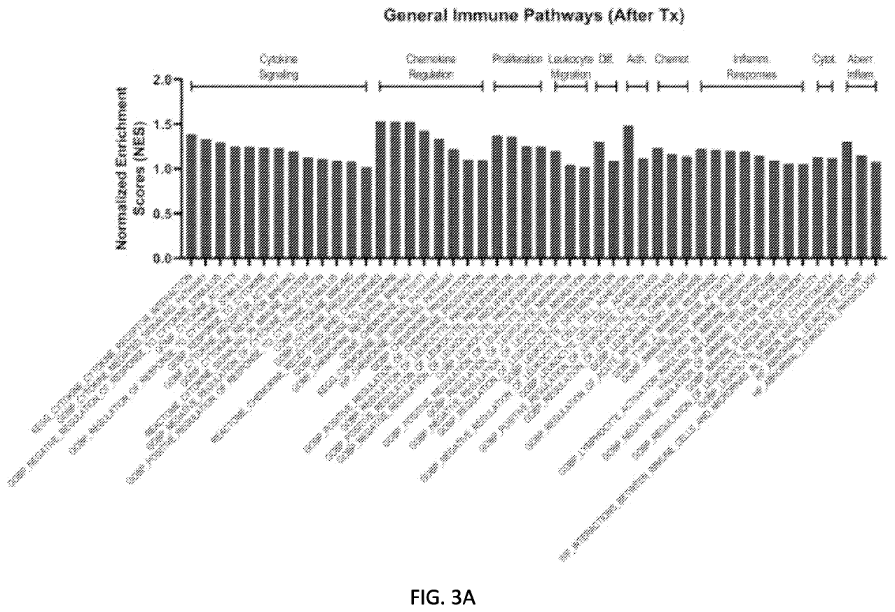

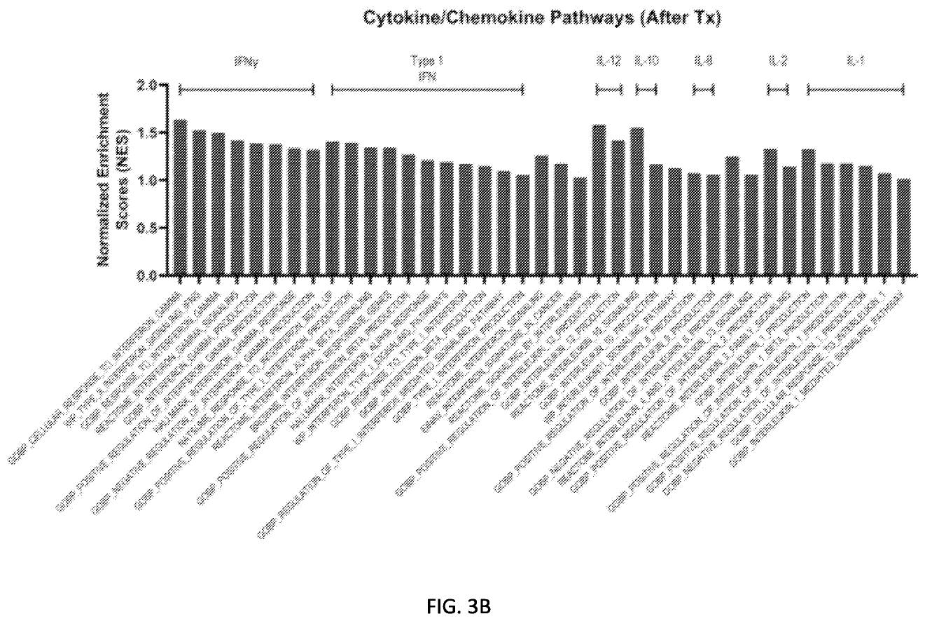

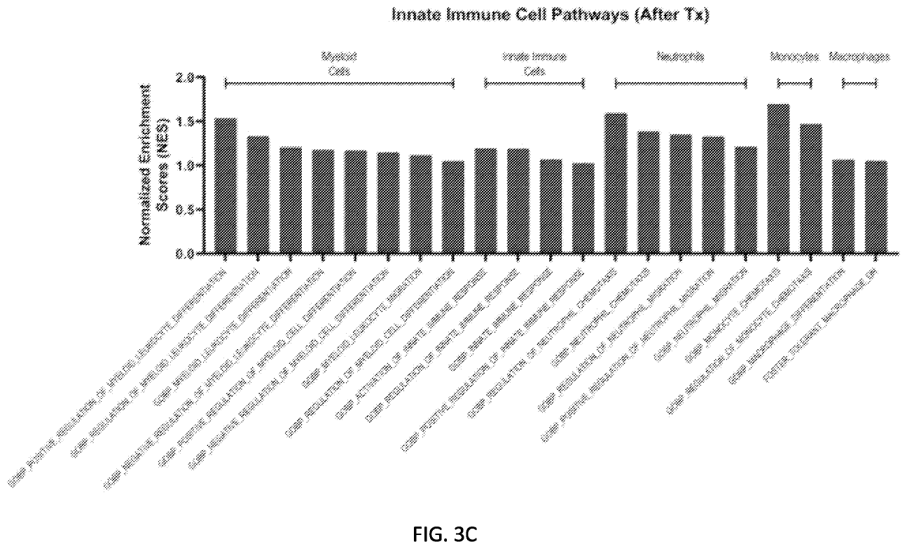

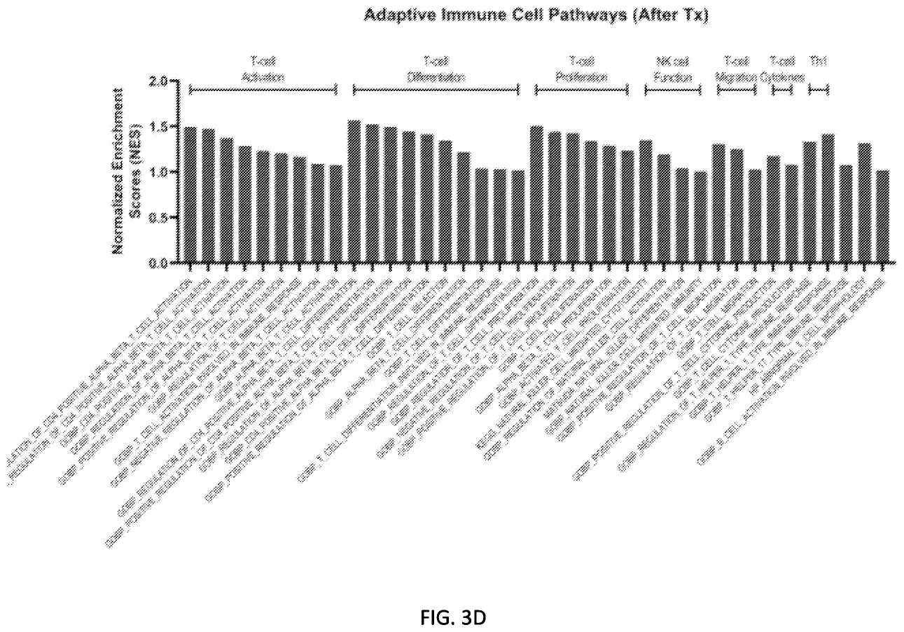

Gene expressions were analyzed with gene set enrichment analysis (GSEA) to assess differentially regulated pathways associated with divergent ICB-responses. GSEA was performed on implant-derived DEseq2-normalized counts from cohorts after Tx. Pathway changes were examined for a total of 4726 identified gene sets. A normalized enrichment score (NES) cutoff of NES>1 was first set to find the most differentially regulated pathways, identifying 2100 pathways above this cutoff. Then, pathways were subcategorized to search for those of the immune system, excluding irrelevant inflammatory pathways. The 143 differentially regulated pathways were broadly categorized into general immune, cytokine/chemokine, myeloid cell, and lymphocyte pathways (FIG. 3). The GSEA analysis of the RNA-seq data identified differentially regulated immune pathways including those associated with cytokine signaling, chemokine regulation, leukocyte proliferation, leukocyte migration, leukocyte differentiation, leukocyte cell-cell adhesion, leukocyte chemotaxis, inflammatory responses, leukocyte cytotoxicity, and aberrant inflammation (FIG. 3A). The most differentially regulated cytokine/chemokine pathways were for those associated with interferon gamma (IFNγ), Type 1 IFN (IFNα, IFNβ), IFN signaling in cancer, interleukin 12 (IL-12), IL-11, IL-10, IL-8, IL-6, IL-4/IL-13, IL-2, and IL-1 (FIG. 3B). The majority of these cytokine/chemokine pathways upregulated in the ICB-sensitive cohort are those responsible for anti-tumor, pro-inflammatory responses. Myeloid cell pathways, including those for neutrophil, monocyte, and macrophage function, were differentially regulated between ICB-sensitive and ICB-resistant (FIG. 3C).

Pathways for innate immune cell chemotaxis, myeloid 82 cell differentiation, and degranulation were downregulated at the immunologic niche of ICB-resistant mice. The most differentially regulated lymphocyte pathways were those associated with T-cell activation, T-cell differentiation, T-cell proliferation, NK cell function, T-cell migration, T-cell cytokine production, Th1 response, Th17 response, aberrant T-cell morphology, B-cell activation (FIG. 3D). Specifically, pro-inflammatory pathways, including activation of T-cells, B-cells, and NK-cells were upregulated in the ICB-sensitive cohort. Much of the downregulated cytokine/chemokine pathways in the ICB-resistant cohort originate from myeloid cells and play a role in suppressing T-cell responses.

In light of the differentially regulated cytokine/chemokine and myeloid cell pathways, it was investigated whether myeloid cell phenotype and function were responsible for differences between the ICB-sensitive and ICB-resistant cohorts at the immunologic niche. Immunologic niche scaffolds (D-14) were implanted, mice were inoculated with 4T1 tumor cells (D0), aPD-1 was administered IP (D9, D11, D13, D15), the engineered niches were explanted a week after completing ICB (D21). ICB-response was determined. The implants were mechanically and enzymatically digested to derive a single cell suspension. Immunologic niche-derived cells were pooled from all mice within either the ICB-sensitive or ICB-resistant cohorts and processed separately. Cells were labelled with magnetic microbead-conjugated anti-CD11b antibodies.

Labelled single cell suspensions were passed through a magnetic activated cell sorting (MACS) column to sort the positive fraction containing CD11b+ myeloid cells. The negative fraction, containing non-myeloid cells such as fibroblasts, was additionally collected. CD11b+ myeloid cell and CD11b-cell suspensions from the ICB-sensitive and ICB-resistant cohorts were lysed in Trizol and RNA isolated. CD11b+ ICB-sensitive, CD11b+ ICB-resistant, CD11b-ICB-sensitive, and CD11b-ICB-resistant RNA was submitted to the AGC for bulk RNA-seq. Results are shown in FIG. 11A-11C.

Following the pathway analyses that showed that T-cell and myeloid cell pathways at the immunologic niche were differentially regulated between ICB-sensitivity and ICB-resistance cohorts after Tx, it was investigated how leukocyte populations were changing more broadly at the immunologic niche, PT, and spleen.

### Ratios of Leukocyte Populations Skewed at Immunologic Niche As Result of Therapy Response

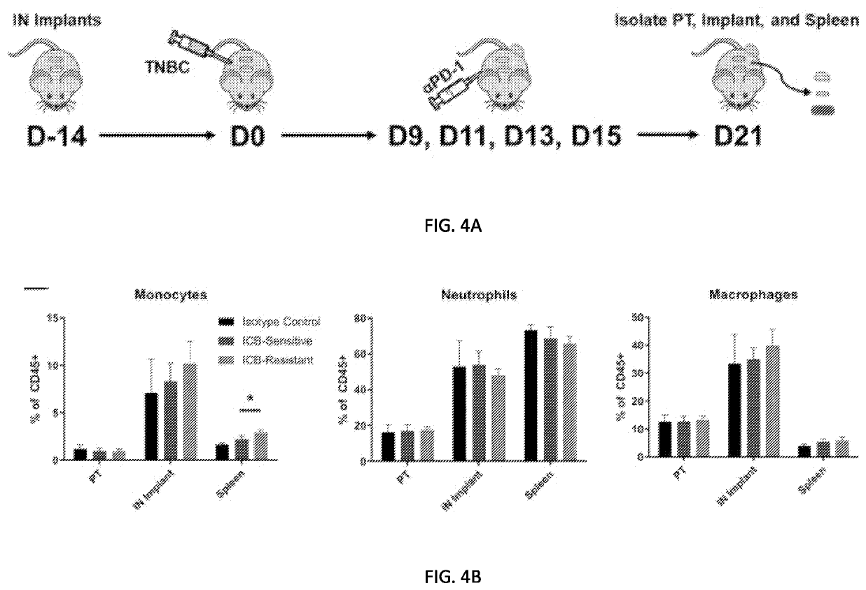

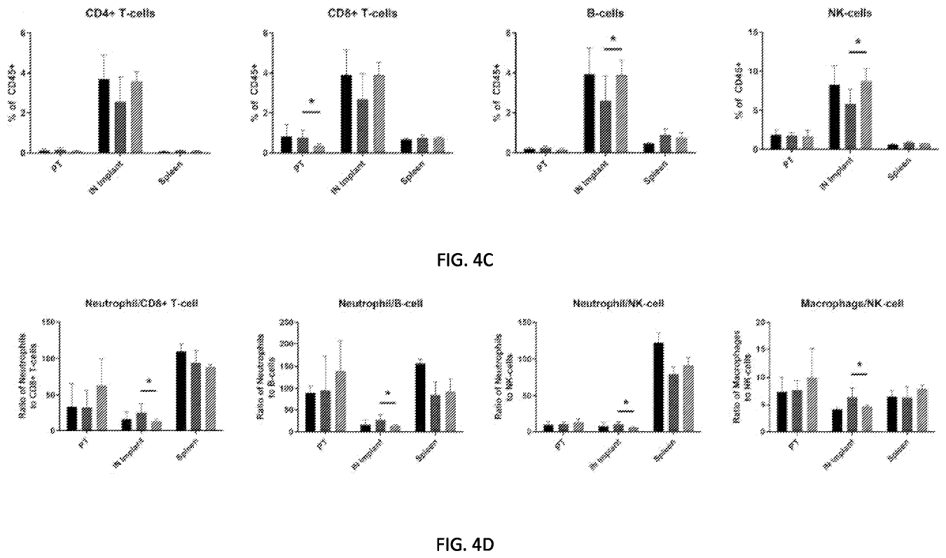

The impact of ICB on systemic immune responses, comparing immune populations at the immunologic niche, PT, and spleen, was next investigated. The PT is regularly biopsied in the clinic and the spleen functions as a surrogate for the blood. Mice with implants were orthotopically inoculated and received aPD-1. Tissues were isolated from mice after Tx (D21) and processed into single cell suspensions. Cells were labelled with fluorophore-conjugated antibodies and analyzed with flow cytometry. No significant difference was observed between the proportion of monocytes (CD11b+Ly6C+), neutrophils (CD11b+Ly6G+), and macrophages (CD11b+F4/80+) at the PTs of ICB-sensitive and resistant mice (FIG. 4B). While some trends were observed, there was no significant difference between the proportions of monocytes, neutrophils, and macrophages at the immunologic niche (IN Implant) of these mice (FIG. 4B). Notably, the PT of ICB-sensitive mice had significantly increased infiltration of cytotoxic T lymphocytes (CD8+) versus the ICB-resistant PTs (FIG. 4C). This observation at the PT is in line with the goal of a T-cell-targeted immunomodulatory therapy. Reduced proportions of both B-cells (CD19+) and NK-cells (CD49b+) were found at the immunologic niche of ICB-sensitive mice (FIG. 4C). Interestingly, the immunologic niche had enriched populations of both myeloid cells (neutrophils, monocytes, macrophages) and lymphocytes (T-cells, B-cells, NK-cells), in comparison to the PT (FIGS. 4B-4C). Ratios of infiltrated myeloid cells to lymphocytes were calculated (FIG. 4D). Excitingly, the ratio of macrophages to NK-cells, neutrophils to NK-cells, neutrophils to B-cells, and neutrophils to T-cells were significantly increased at the immunologic niche of ICB-sensitive mice versus ICB-resistant mice (FIG. 4D). Differences in the myeloid cell to lymphocyte ratio, between the ICB-sensitive and ICB-resistant cohorts, was not observed at the PT and spleen. Following the observation that the engineered implants capture unique immune cell dynamics after Tx, and that implant-derived genes can be monitored for ICB-response, it was investigated whether the immunologic niche could be probed before Tx for predictive gene expressions.

### Gene Expressions Predictive of ICB-Response

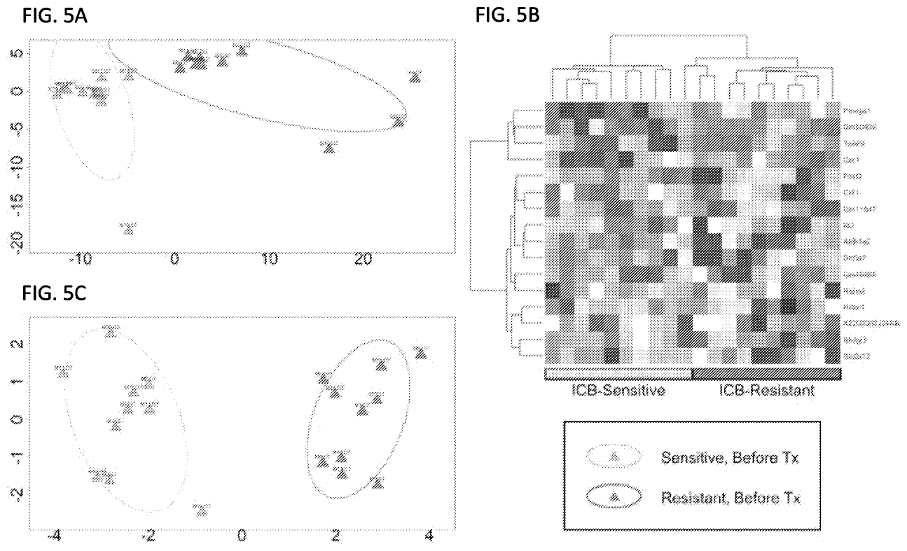

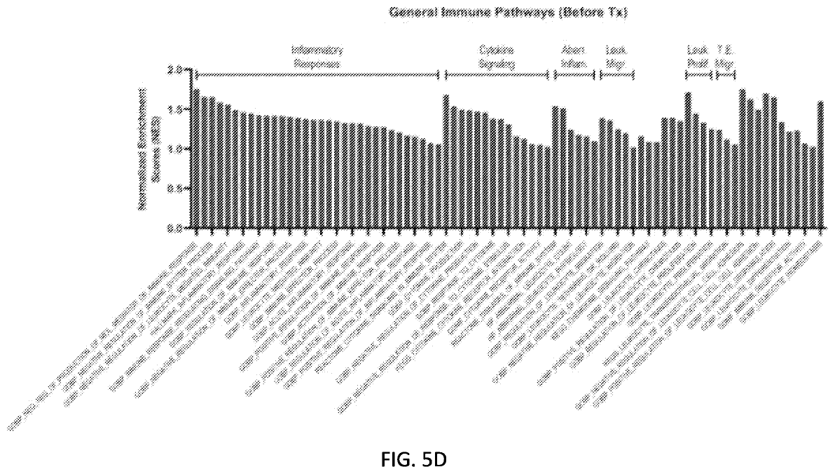

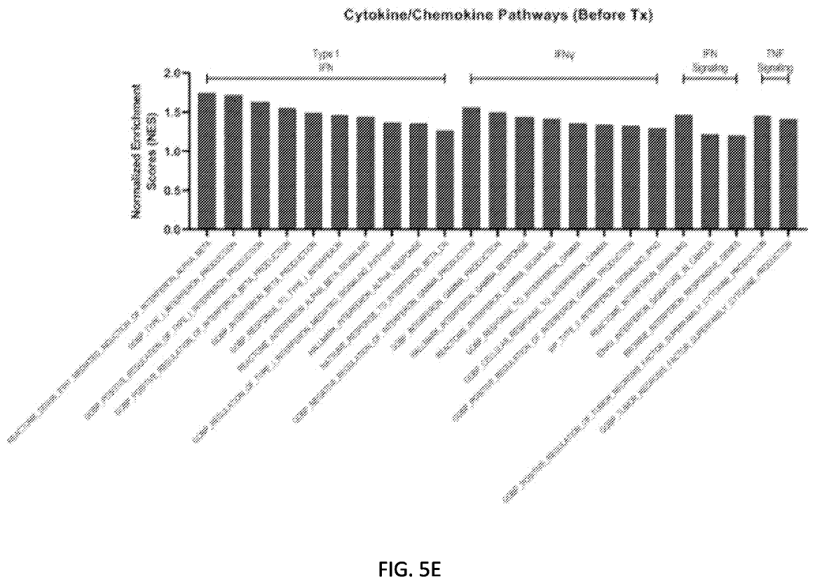

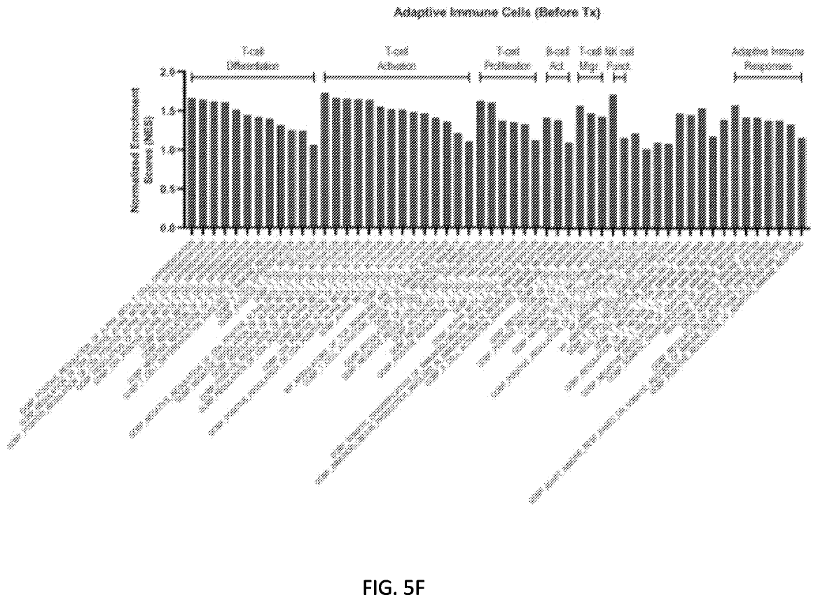

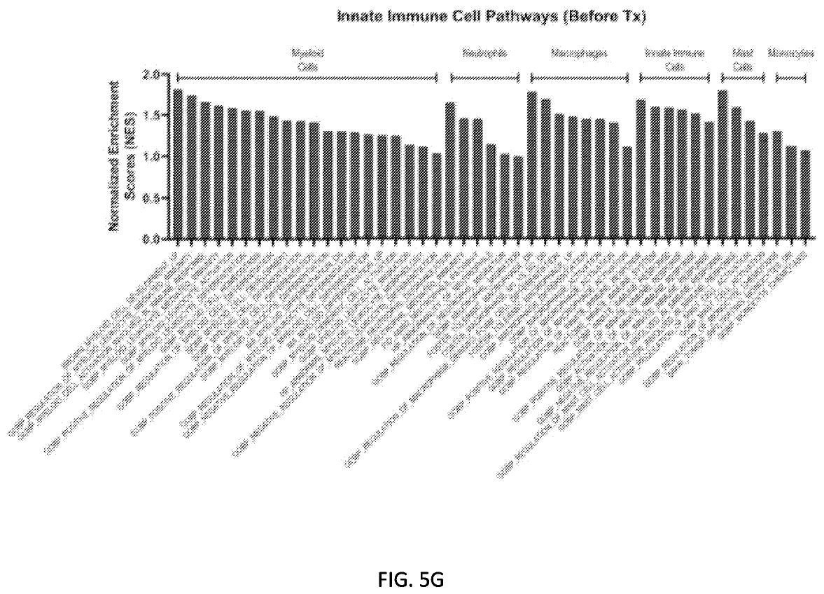

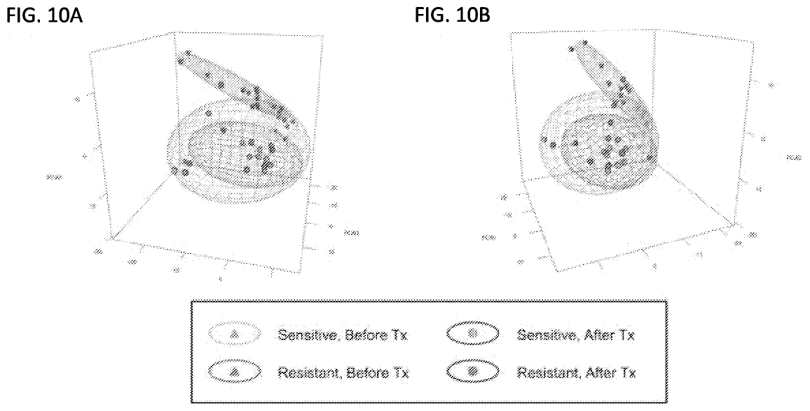

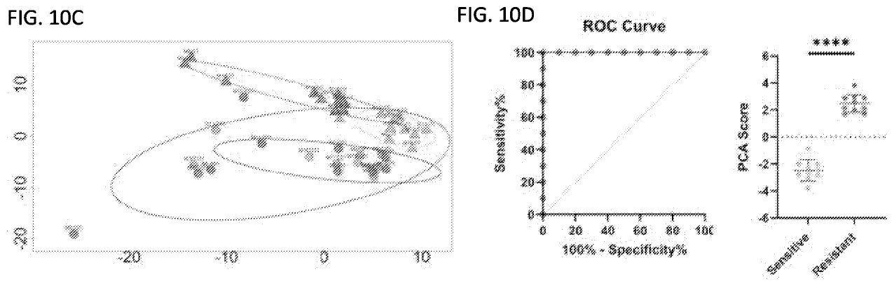

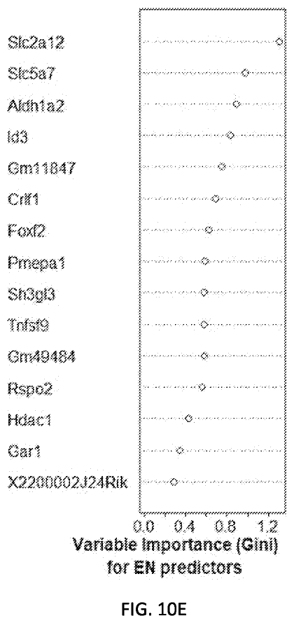

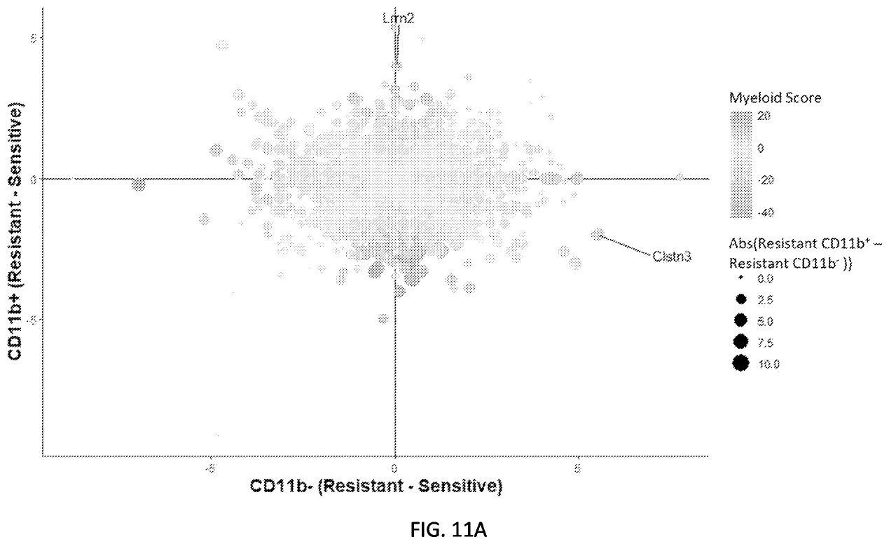

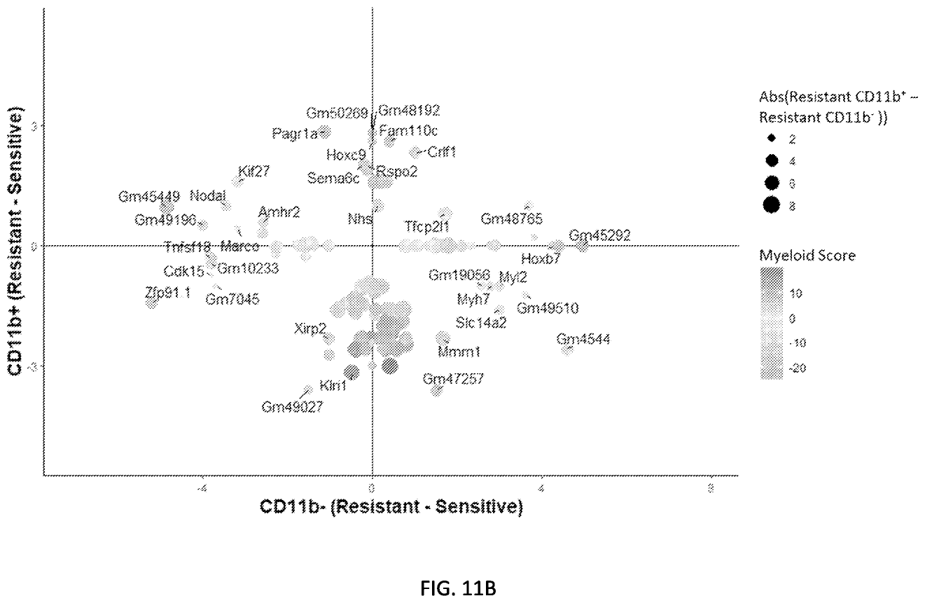

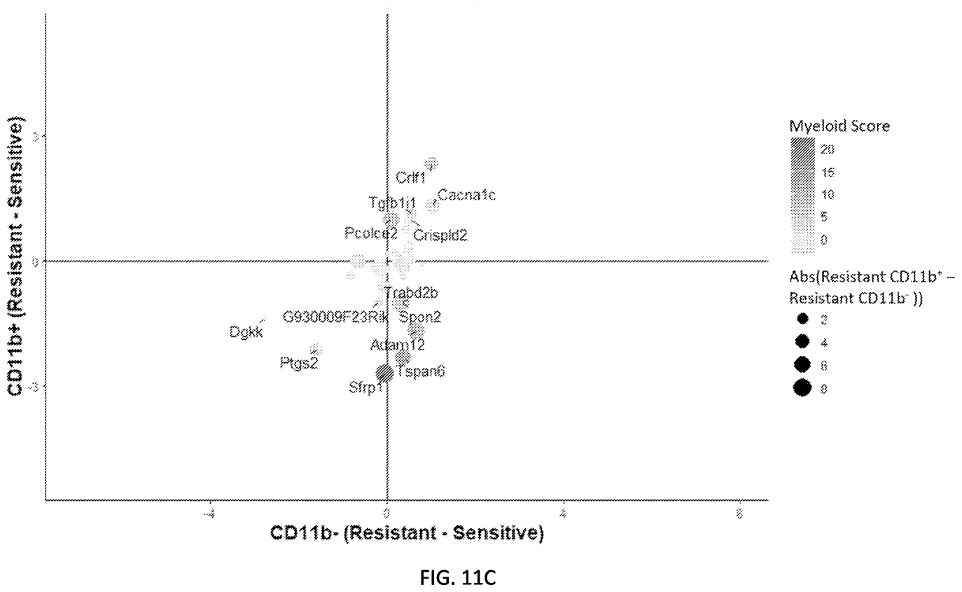

The delta analysis showed that dynamic gene expression changes can be monitored at the implant for ICB-response (FIG. 2). Interestingly, the PCA-based clustering of the DEseq2-normalized counts from the delta panel showed mildly separate clustering between ICB-sensitive and resistant before Tx (FIG. 2B). As such, it was investigated whether a unique set of predictive genes could be identified before initiating Tx (D7). The DEseq2-normalized counts before Tx (D7) were first screened with T-tests between the gene expression of ICB-sensitive and resistant, identifying 331 DEGs (FIG. 5A). PCA of these before Tx DEGs showed differential clustering of sensitive and resistant cohorts (FIG. 5A). EN was them employed to identify a 16-gene panel of DEGs before Tx. Clustering of the implant-derived gene expressions from the before Tx-panel showed superb categorization of ICB-sensitivity and resistance prior to administering ICB (FIG. 5C). Notably, 12/16 genes were upregulated in the mice, before Tx, that would become sensitive to ICB and hierarchical clustering categorized the cohorts based on predicted ICB-response (FIG. 5B). Performance metrics were calculated for sensitivity, specificity, and categorization efficacy (FIG. 10D). The 16 gene panel was found to predict ICB sensitive and ICB resistant patients with a high sensitivity and specificity. Interestingly, the categorization metrics indicated that the before Tx gene signature more effectively predicted ICB-response than the delta gene signature for monitoring ICB-response (FIG. 9C). Based on the exciting observations that a multivariate panel of genes was predictive of ICB-response prior to Tx, differentially regulated pathways underlying these differences were investigated. GSEA was performed on the implant-derived gene expressions before Tx. Differentially regulated pathways were examined for 4880 identified gene sets. An NES>1 cutoff was used to identify the 2671 most differentially regulated pathways. Immune system-associated pathways were then subcategorized, excluding irrelevant inflammatory pathways. The 207 differentially regulated immune pathways were broadly categorized into general immune, cytokine/chemokine, myeloid cell, and lymphocyte pathways (FIGS. 5D-5G) . The 81 general immune system pathways differentially regulated between ICB-sensitivity and resistance included pathways associated with inflammatory responses, cytokine signaling, aberrant inflammation, migration, chemokine regulation, chemotaxis, proliferation, leukocyte transendothelial (T.E) migration, leukocyte cell-cell adhesion, leukocyte degranulation, leukocyte differentiation, immune receptor signaling, leukocyte cytotoxicity, and leukocyte homeostasis (FIG. 5D). The 23 most differentially regulated cytokine/chemokine pathways included those associated with Type 1 IFN (IFNα, IFNβ), IFNγ, and TNF signaling (FIG. 5E). The 47 myeloid cell-associated pathways included those of neutrophils, macrophages, innate immune cell responses, mast cells, and monocytes (FIG. 5F). Interestingly, pathways including myeloid cell homeostasis, myeloid cell differentiation, and neutrophil-mediated immunity were upregulated, whereas macrophage tolerance and macrophage M1 vs M2 pathways were downregulated at the implant before Tx (FIG. 5F). Finally, the most differentially regulated lymphocyte pathways between ICB-sensitive and ICB-resistant before Tx were pathways associated with T-cell differentiation, T-cell activation, T-cell proliferation, B-cell activation, T-cell migration, NK cell function, T-cell cytokine production, aberrant T-cell function, T-cell receptor signaling, Th1 response, Th17 response, and adaptive immune cell responses (FIG. 5G). Some of these pathways upregulated at the immunologic niche of ICB-sensitive mice before Tx included modulators of T-cell receptor signaling (TCR) and the somatic diversification of immunoglobulins involved in immune responses (FIG. 5G). Collectively, these studies point to the clinical utility of the immunologic niche for 1) predicting ICB-response prior to initiating therapy, 2) monitoring ICB-sensitivity during therapy, and 3) illuminating the role of differentially regulated immune cell pathways responsible for divergent ICB-responses.

### Discussion

In this study, the immunologic niche was investigated as a diagnostic for ICB-response. While checkpoint inhibitors have been approved 1) as a treatment for metastatic TNBC and 2) as neoadjuvant/adjuvant therapies surrounding TNBC PT resection, and profound results have been elicited in some patients, the majority are resistant to ICB. The utility of ICB in TNBC has been greatly limited by the clinical needs to 1) identify ICB-sensitivity and resistance prior to treatment and 2) monitor dynamic responses to ICB. Much of this is a result of the inherent limitations of a PT biopsy, representing a restricted snapshot of biomarkers confined to the local microenvironment. Liquid biopsy-derived biomarkers have shown some clinical utility in monitoring CTC burden and identifying chemotherapy options, however they have shown insufficient success in identifying immunotherapy-associated biomarkers. This may be, in part, because of differences in immune cell phenotype and function between those in the blood and those that have extravasated into a tissue.

The immunologic niche investigated herein provides a unique opportunity to study immune cells that have extravasated systemic vasculature into a tissue that can be longitudinally probed. It is demonstrated herein that the implant has enriched populations of immune cells versus the PT and that implant-derived gene expressions are predictive of ICB-response prior to therapy (FIGS. 4, 5). These gene expressions can be probed to glean insight into differentially regulated pathways, which may provide additional insight into ICB-resistance-associated mechanisms (FIGS. 4, 5). This is especially exciting, given that multimodal, combination therapies have been shown to increase ICB response rates. Insight into dysregulated immune pathways underlying ICB-resistance provides a basis for selecting chemo-, immuno-, or radiation therapies to skew ICB-resistant patients as an induction strategy prior to ICB.

Biomarker expression at a PT is often discordant with biomarker expression at metastatic foci, which provides significant complications when making clinical decisions from PT-derived biomarkers, especially when treatments for metastatic BC are informed by a PT biopsy that may have been collected many years prior. In BC, metastatic foci are typically localized to the lungs, liver, bone marrow, or brain. As a result of the anatomical constraints of these tissues, radiographic imaging is the gold standard for monitoring therapy response in treating metastatic disease. For the majority of chemo-and radiotherapies, change in metastatic lesion size is indicative of therapy response, where a decrease in the size of a lesion indicates therapy sensitivity. Pseudoprogression and hyperprogression following ICB therapy are unique phenomena that confound the ability to correlate lesion size with immunotherapy response.

Pseudoprogression is characterized by the radiographic appearance of lesion growth, as a result of immune cell infiltration in ICB-sensitive patients. This initial growth is subsequently followed by tumor regression as a result of an anti-tumor immune response. The inability to delineate between true progression of disease, as a result of ICB-resistance, and pseudoprogression among ICB-sensitive patients highlights the clinical need for a technology to profile the immune responses to ICB. The immunologic niche was shown to probe immune system dynamics resulting from ICB, and implant-derived gene expressions could be monitored for ICB-response both during and after ICB (FIGS. 2, 3, 9D). This technology can be used in conjunction with radiographic imaging for monitoring ICB-response. Additionally, a small proportion of patients experience hyperprogression, or a rapid progression of disease following the initiation of ICB.

Radiographic imaging alone cannot differentiate pseudoprogression from hyperprogression when lesion enlargement is observed. The phenomena of pseudoprogression and hyperprogression emphasize the clinical need to predict ICB-response prior to therapy. The standard of care for predicting ICB-response has been limited to PD-L1 expression, leukocyte infiltration, and tumor mutational burden. Clinical trials have found improved response rates among BC patients with high TMB and TNBC patients with PD-L1+ tumors, however this is not predictive of ICB-response and has performed poorly at identifying ICB-sensitive patients.

Selecting TNBC patients with PD-L1+ tumor infiltrating lymphocytes has identified specific subpopulations that have the best ICB-response, but even the majority of this subpopulation is ICB-resistant. The FDA approved the use of ICB in early TNBC, in the neoadjuvant/adjuvant setting surround PT resection. This setting has the goal of first reducing PT volume with neoadjuvant ICB, prior to resection, followed by adjuvant ICB to mitigate recurrence. Not all patients require adjuvant ICB, and as of yet, there is no way to stratify patients into those that would benefit from ICB following surgical resection. Taking the adverse side effects associated with ICB and cost to the medical system into account, there is a great clinical need for identifying which patients would benefit from ICB. Analysis of implant-derived gene expressions prior to ICB provided multivariate analytes predictive of ICB-response (FIG. 4-5). Probing these gene expressions before Tx also identified differentially regulated myeloid cell pathways, suggesting myeloid cells may play a role in ICB-resistance (FIG. 4-5).

In conclusion, the ability to longitudinally biopsy an accessible site to monitor ICB-response-associated biomarkers in real time, as described herein, is a viable and useful tool for managing metastatic disease.

We claim:

1. A method comprising:
a) obtaining a sample from a microenvironment of a synthetic scaffold implanted in a subject, wherein the subject is a candidate for treatment with an immune checkpoint inhibitor or has received treatment with an immune checkpoint inhibitor, and
b) measuring an expression level or amount of at least one RNA, at least one gene, at least one cell type, and/or at least one protein in the sample.

2. The method of claim 1 or claim 2, wherein the subject has or is suspected of having breast cancer.

3. The method of claim 2, wherein the breast cancer is triple negative breast cancer (TNBC).

4. The method of any one of claims 1-3, wherein the subject has received treatment with an immune checkpoint inhibitor, and wherein the sample is obtained from the microenvironment of the synthetic scaffold 1-28 days after receiving the treatment.

5. The method of claim 4, wherein the sample is obtained from the microenvironment of the synthetic scaffold 7-21 days after receiving the treatment.

6. A method comprising:
a) obtaining a sample from a microenvironment of a synthetic scaffold implanted in a subject, wherein the subject is diagnosed with or at risk of having cancer and wherein the subject has not received treatment with an immune checkpoint inhibitor, and
b) measuring an expression level of a panel of genes in the sample, wherein the panel of genes comprises two or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

7. The method of claim 6, wherein the panel of genes comprises Slc2a12, Slc5a7, Aldh1a2, and Id3.

8. The method of claim 6 or claim 7, wherein the panel of genes comprises six or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

9. The method of claim 8, wherein the panel of genes comprises 10 or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

10. The method of claim 9, wherein the panel of genes comprises each of Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

11. The method of any one of claims 6-10, wherein the panel of gene comprises less than 50 genes.

12. The method of any one of claims 6-11, further comprising:
a) providing an immune checkpoint inhibitor to the subject when the expression level of one or more of Pmepa11, Gm50439, Tnfsf9, and Gar1is decreased relative a control level or a cutoff level and/or when the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level; or
b) providing an alternative cancer treatment to the subject when the expression level of one or more of Pmepa11, Gm50439, Tnfsf9, and Gar1is increased relative a control level or a cutoff level and/or the expression level of one or more of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level.

13. The method of any one of claims 6-12, wherein the cancer is breast cancer.

14. The method of claim 13, wherein the cancer is triple negative breast cancer.

15. A method of predicting response to an immune checkpoint inhibitor in a subject, the method comprising:
a) obtaining a sample from a microenvironment of a synthetic scaffold implanted in a subject, wherein the subject is diagnosed with or at risk of having cancer and is a candidate for treatment with an immune checkpoint inhibitor;
b) measuring an expression level or amount of at least one RNA, at least one gene, at least one cell type, and/or at least one protein in the sample; and
c) determining whether the subject is likely to be sensitive or resistant to treatment with the immune checkpoint inhibitor based upon the expression level or amount of the at least one RNA, the at least one gene, the at least one cell type, and/or the at least one protein in the sample.

16. The method of claim 15, comprising measuring the expression level or amount of a panel of RNA, a panel of genes, a plurality of cell types, and/or a panel of proteins in the sample.

17. The method of claim 15 or claim 16, wherein the cancer is breast cancer.

18. The method of claim 17, wherein the cancer is triple negative breast cancer (TNBC).

19. The method of any one of claims 15-18, wherein the subject has not received treatment with an immune checkpoint inhibitor.

20. The method of any one of claims 15-19, further comprising comparing the expression level or amount of the at least one RNA, at least one gene, at least one cell type, and/or at least one protein in the sample to a control level or a cutoff level.

21. The method of any one of claims 15-20, comprising measuring the expression level of one or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

22. The method of claim 21, wherein the panel of genes comprises Slc2a12, Slc5a7, Aldh1a2, and Id3.

23. The method of claim 21 or claim 22, comprising measuring the expression level of six or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

24. The method of claim 23, comprising measuring the expression level of 10 or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

25. The method of claim 24, comprising measuring the expression level of each of Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

26. The method of any one of claims 15-25, further comprising:
a) determining that the subject is likely to be sensitive to treatment with the immune checkpoint inhibitor when the expression level of one or more of Pmepa11, Gm50439, Tnfsf9, and Gar1is decreased relative a control level or a cutoff level;
b) determining that the subject is likely to be sensitive to treatment with the immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is increased relative to a control level or a cutoff level;
c) determining that the subject is likely to be resistant to treatment with the immune checkpoint inhibitor when the expression level of one or more of Pmepa11, Gm50439, Tnfsf9, and Gar1is increased relative a control level or a cutoff level; and/or
d) determining that the subject is likely to be resistant to treatment with the immune checkpoint inhibitor when the expression level of one or more of Foxf2, Crlf1, Gm11847, Id3, Aldh1a2, Slc5a7, Gm49484, Rspo2, Hdac1, X2200002j24Rik, Sh3gf3, and Slc2a12 is decreased relative to a control level or a cutoff level.

27. The method of any one of claims 15-26, further comprising providing the immune checkpoint inhibitor to the subject determined as likely to be sensitive to treatment with the immune checkpoint inhibitor, or providing an alternative cancer treatment to the subject identified as being likely to be resistant to treatment with the immune checkpoint inhibitor.

28. A method comprising:
a) obtaining a sample from a microenvironment of a synthetic scaffold implanted in a subject, wherein the subject is diagnosed with or at risk of having cancer and wherein the subject has received treatment with an immune checkpoint inhibitor; and
b) measuring an expression level of a panel of genes in the sample; and/or
c) measuring an amount of one or more cell types selected from B-cells, NK-cells, neutrophils, T-cells, and macrophages in the sample.

29. The method of claim 28, wherein the cancer is breast cancer.

30. The method of claim 29, wherein the cancer is triple negative breast cancer (TNBC).

31. The method of any one of claims 28-30, wherein the sample is obtained from the microenvironment of the synthetic scaffold 1-28 days after receiving treatment with the immune checkpoint inhibitor.

32. The method of claim 31, wherein the sample is obtained from the microenvironment of the synthetic scaffold 7-21 days after receiving treatment with the immune checkpoint inhibitor.

33. The method of any one of claims 28-32, further comprising determining whether the subject is sensitive or resistant to treatment with the immune checkpoint inhibitor based upon the expression level of the panel of genes and/or the amount of the one or more cell types in the sample.

34. The method of claim 33, further comprising providing an alternative treatment to the subject determined to be resistant to treatment with the immune checkpoint inhibitor.

35. The method of claim any one of claims 28-34, wherein the panel of genes comprises:
a) two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nup11, Tlr12, and Alms1;
b) two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3; or
c) two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2.

36. The method of claim 35, wherein the panel of genes comprises six or more genes.

37. The method of claim 36, wherein the panel of genes comprises 10 or more genes.

38. The method of any one of claims 36-37, wherein the panel of gene comprises less than 50 genes.

39. The method of any one of claims 36-38, comprising measuring an amount of one or more cell types selected from B-cells, NK-cells, neutrophils, T-cells, and macrophages in the sample, and:
a) providing an alternative cancer treatment to the subject when the amount of B-cells is increased relative to a control level or a cutoff level;
b) providing an alternative cancer treatment to the subject when the amount of NK-cells is increased relative to a control level or a cutoff level; or
c) providing an alternative cancer treatment to the subject when a ratio of the amount of neutrophils: T-cells, neutrophils: B-cells, neutrophils: NK-cells, and/or macrophages: NK-cells is decreased relative to a control.

40. A method of monitoring response to treatment with an immune checkpoint inhibitor in a subject, the method comprising:
a) obtaining a sample from a microenvironment of a synthetic scaffold implanted in a subject; and
b) measuring an expression level or amount of at least one RNA, at least one gene, at least one cell type, and/or at least one protein in the sample,
wherein the subject is diagnosed with or at risk of having cancer, and wherein the subject has received treatment with an immune checkpoint inhibitor.

41. The method of claim 40, wherein the sample is obtained from the microenvironment of the synthetic scaffold 1-28 days after receiving the treatment.

42. The method of claim 41, wherein the sample is obtained from the microenvironment of the synthetic scaffold 7-21 days after receiving the treatment.

43. The method of any one of claims 40-42, further comprising comparing the expression level or amount of the at least one RNA, at least one gene, at least one cell type, and/or at least one protein in the sample to a control level or a cutoff level.

44. The method of claim 43, further comprising determining whether the subject is sensitive or resistant to treatment with the immune checkpoint inhibitor based upon the expression level or amount of the at least one RNA, at least one gene, at least one cell type, and/or at least one protein in the sample.

45. The method of any one of claims 40-44, comprising:
a) measuring the expression level of two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nup11, Tlr12, and Alms1;
b) measuring the expression level of two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3;
c) measuring the expression level of two or more genes selected from Chsy1, Sprtn, Gm18935, Timm44, Gm19056, Rp118.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2; and/or
d) measuring the amount of one or more cell types selected from B-cells, NK-cells, neutrophils, T-cells, and macrophages.

46. The method of claim 45, comprising performing step a), and:
i) determining that the subject is sensitive to the treatment with the immune checkpoint inhibitor when the expression level of one or more of Hs6st2, Gm48732, Gm50393, Gbp2, Rtp4, and Gbp3 is increased relative a control level or a cutoff level;
ii) determining that the subject is resistant to the treatment with the immune checkpoint inhibitor when the expression level of one or more of Hs6st2, Gm48732, Gm50393, Gbp2, Rtp4, and Gbp3 is decreased relative a control level or a cutoff level;
iii) determining that the subject is sensitive to the treatment with the immune checkpoint inhibitor when the expression level of one or more of Tpsab1, Surf1. Rnf144a, Stfa2, Crlf1, Sprtn, Sh3g13, Nupl1, Tlr12, and Alms1 is decreased relative to a control level or a cutoff level; or
iv) determining that the subject is resistant to the treatment with the immune checkpoint inhibitor when the expression level of one or more of Tpsab1, Surf1. Rnf144a, Stfa2, Crlf1, Sprtn, Sh3g13, Nupl1, Tlr12, and Alms1 is increased relative to a control level or a cutoff level.

47. The method of claim 45, comprising performing step b), and:
i) determining that the subject is sensitive to the treatment with the immune checkpoint inhibitor when the expression level of one or more of Slc7a14, Dkk2, Fdps, Trib1, Pagr1a, Csf1, Ptgs2, Gm50393, H2.M2, Gm19195, Sycp2, X2810457G06Rik, and Gm48996 is increased relative to a control level or a cutoff level,
ii) determining that the subject is resistant to the treatment with the immune checkpoint inhibitor when the expression level of one or more of Slc7a14, Dkk2, Fdps, Trib1, Pagr1a, Csf1, Ptgs2, Gm50393, H2.M2, Gm19195, Sycp2, X2810457G06Rik, and Gm48996 is decreased relative to a control level or a cutoff level,
iii) determining that the subject is sensitive to the treatment with the immune checkpoint inhibitor when the expression level of one or more of Slc4a4, Dtd2, Lvrn, Rnf144a, Zic4, Nupl1, Cd177, Stfa2, and Ripply3 is decreased relative to a control level or a cutoff level; or
iv) determining that the subject is resistant to the treatment with the immune checkpoint inhibitor when the expression level of one or more of Slc4a4, Dtd2, Lvrn, Rnf144a, Zic4, Nupl1, Cd177, Stfa2, and Ripply3 is increased relative to a control level or a cutoff level.

48. The method of claim 45, comprising performing step c), and:
i) determining that the subject is sensitive to the treatment with the immune checkpoint inhibitor when the expression level of one or more of Gm45290, Timm44, Gm19056, Pla2g12a, Gm9118, Lhx6, Tex22, 8430429K09Rik, Gm6361, 530010L16Rik, Gm18362, and Ptgs2 is increased relative to a control level or a cutoff level;
ii) determining that the subject is resistant to the treatment with the immune checkpoint inhibitor when the expression level of one or more of Gm45290, Timm44, Gm19056, Pla2g12a, Gm9118, Lhx6, Tex22, 8430429K09Rik, Gm6361, 530010L16Rik, Gm18362, and Ptgs2 is decreased relative to a control level or a cutoff level;
iii) determining that the subject is sensitive to the treatment with the immune checkpoint inhibitor when the expression level of one or more of Chsy1, Sprint, Gm18935, Rpl18.ps2, Tlr12, 2900078111Rik, Crlf1, Wdr66, and Syde22 is decreased relative to a control level or a cutoff level; or
iv) determining that the subject is resistant to the treatment with the immune checkpoint inhibitor when the expression level of one or more of Chsy1, Sprint, Gm18935, Rpl18.ps2, Tlr12, 2900078111Rik, Crlf1, Wdr66, and Syde22 is increased relative to a control level or a cutoff level.

49. The method of claim 45, comprising performing step d), and:
i) determining that the subject is resistant to the treatment with the immune checkpoint inhibitor when the amount of B-cells is increased relative to a control level or a cutoff level;
ii) determining that the subject is resistant to the treatment with the immune checkpoint inhibitor when the amount of NK-cells is increased relative to a control level or a cutoff level; or
iii) determining that the subject is resistant to the treatment with the immune checkpoint inhibitor when a ratio of the amount of neutrophils: T-cells, neutrophils: B-cells, neutrophils: NK-cells, and/or macrophages: NK-cells is decreased relative to a control.

50. The method of any one of claims 40-49, further comprising providing an alternative cancer treatment to the subject identified as resistant to treatment with the immune checkpoint inhibitor.

51. A kit comprising reagents for detection of a panel of genes in a sample, the panel of genes comprising two or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

52. The kit of claim 51, wherein the panel of genes comprises six or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

53. The kit of claim 52, wherein the panel of genes comprises 10 or more genes selected from Pmepa1, Gm50439, Tnfsf9, Gar1, Foxf2, Crlf1, Gm11847, Id3, Aldh1a2, Slc5a7, Gm49484, Rspo2, Hdac1, X2200002j24Rik, Sh3gf3, and Slc2a12.

54. The kit of any one of claims 51-53, wherein the panel of genes comprises less than 50 genes.

55. The kit of any one of claims 51-54, for use in a method of predicting response to an immune checkpoint inhibitor in a subject.

56. The kit of claim 55, for use in a method of predicting response to an immune checkpoint inhibitor in a subject prior to administration of the immune checkpoint inhibitor to the subject.

57. A kit comprising reagents for detection of a panel of genes in a sample, the panel of genes comprising:
a) two or more genes selected from Hs6st2, Gm48732, Gm50393, Gbp2, Rtp4, Gbp3, Tpsab1, Surf1, Rnf144a, Stfa2, Crlf1, Sprtn, Sh3g13, Nupl1, Tlr12, and Alms1;
b) two or more genes selected from Slc7a14, Dkk2, Fdps, Trib1, Pagr1a, Csf1, Ptgs2, Gm50393, H2.M2, Gm19195, Sycp2, X2810457G06Rik, Gm48996, Slc4a4, Dtd2, Lvrn, Rnf144a, Zic4, Nupl1, Cd177, Stfa2, and Ripply3; or
c) two or more genes selected from Chsy1, Sprtn, Gm18935, Timm44, Gm19056, Rpl18.ps2, Tlr12, X2900078111Rik, Crlf1, Wdr66, Pla2g12a, Gm9118, Syde2, A530010L16Rik, Gm183562, Lhx6, Tex22, Grip1, X8430429K09Rik, Gm6361, and Ptgs2.

58. The kit of claim 57, wherein the panel of genes comprises six or more genes.

59. The kit of claim 58, wherein the panel of genes comprises 10 or more genes.

60. The kit of any one of claims 57-59, wherein the panel of genes comprises less than 50 genes.

61. The kit of any one of claims 57-60, for use in a method of monitoring response to a treatment with an immune checkpoint inhibitor in a subject.

62. The kit of claim 61, wherein monitoring response to the treatment with the immune checkpoint inhibitor in the subject comprises contacting a sample obtained from a microenvironment of a synthetic scaffold implanted in the subject with the reagents for detection of the panel of genes.

63. The kit of claim 62, wherein the sample is obtained about 1-28 days after receiving the treatment with the immune checkpoint inhibitor.

64. The kit of claim 63, wherein the sample is obtained about 7-21 days after receiving the treatment with the immune checkpoint inhibitor.
